(19) United States
(12) Patent Application Publication
(10) Pub. No.: US 2014/0261425 A1
(43) Pub. Date: Sep. 18, 2014

(54) Wearable Positive Airway Pressure (PAP) with Primary and Bolus Airflow

(71) Applicant: Robert A. Connor, Forest Lake, MN (US)

(72) Inventor: Robert A. Connor, Forest Lake, MN (US)

(21) Appl. No.: 13/801,007

(22) Filed: Mar. 13, 2013

(51) Int. Cl. A61M 16/00

(52) U.S. Cl. CPC A61M 16/0057; A61M 16/0051
USPC 128/204.23

(57) ABSTRACT

This invention can be embodied as a wearable self-contained device that provides energy-efficient Positive Airway Pressure (PAP) to treat Obstructive Sleep Apnea (OSA). This device includes a primary airflow member and a bolus airflow member which operate together in a coordinated manner to provide just the right amount of pressure that is needed at any given time in order to keep the person's airway open in the most energy-efficient manner. The bolus airflow member can accumulate energy over multiple respiratory cycles and use this energy to release a bolus of air when needed. Energy efficiency is particularly critical for wearable self-contained Positive Airway Pressure (PAP) systems that a person can wear on their head without a tube connection to a bedside unit or a continuous wire connection to an external power source.

### CROSS-REFERENCE TO RELATED APPLICATIONS

This patent application claims the priority benefit of U.S. Provisional Patent Application No. 61/736,532 entitled “Wearable Positive Airway Pressure (PAP) with Primary and Bolus Airflow” filed on Dec. 12, 2012 by Robert A. Connor of Sleepnea, LLC.

### FEDERALLY SPONSORED RESEARCH

Not Applicable

### SEQUENCE LISTING OR PROGRAM

Not Applicable

### BACKGROUND

### Field of Invention

This invention relates to positive airway pressure for Obstructive Sleep Apnea (OSA).

### INTRODUCTION TO OBSTRUCTIVE SLEEP APNEA (OSA) AND AIR PUMP ENERGY EFFICIENCY

Obstructive Sleep Apnea (OSA) is intermittent blockage of a person's airway while they sleep. Such blockages can occur hundreds of times each night, causing poor sleep and oxygen deprivation. Obstructive sleep apnea can cause serious long-term harmful effects. These harmful effects include: disrupted sleep; chronic fatigue; morning headaches; irritability; brain damage; cognitive dysfunction; impotency; high blood pressure; heart attacks; congestive heart failure; motor vehicle crashes; job-site accidents; and even death. Despite these harmful effects, it is estimated that only 5% to 8% of the affected population are treated. Approximately 20 million Americans and 35 million people worldwide have obstructive sleep apnea and the number is growing rapidly.

Positive airway pressure is a common and effective therapy for OSA. Positive airway pressure can help to keep a person's soft tissue from collapsing into their airway during sleep. This keeps the airway open during sleep, particularly before or during the onset of inhalation when collapse is most likely to occur. Variations on positive airway pressure include Continuous Positive Airway Pressure (CPAP) and Positive End Expiratory Pressure (PEEP). CPAP provides virtually continuous positive airway pressure. PEEP provides positive airway pressure at selected times when airway collapse is most likely to occur. Positive airway pressure can be provided by an active energy-using air-moving device such as an electricity-powered air pump. Air from such a pump is channeled into a person's airway via an air hose into a mask or nasal inserts which a person wears while they sleep. Generally, an air pump is incorporated into a bedside unit which is connected to a mask or nasal inserts by an air hose.

However, many people with Obstructive Sleep Apnea (OSA) who would benefit from positive airway pressure do not use it. Most of these people cannot tolerate wearing a sleep apnea mask. For some, wearing such a mask on their face makes them feel claustrophobic and they cannot sleep wearing one. Other people cannot tolerate being tethered to a bedside air pump. They get tangled up in the air hose while they toss and turn in their sleep. Other people who would benefit from positive airway pressure cannot use it because they live in areas of the world that lack access to continuous dependable electrical power.

For these reasons, there have been efforts to create self-contained head-worn positive airway pressure units that combine an air pump and mask into a single, battery-powered positive airway pressure unit that a person wears on their head. The goal is to have no air hose tethering the person to a bedside pump and no power wire connecting the person to an electrical wall outlet. Such a self-contained head-worn positive airway pressure unit can free a person from being tethered to a bedside air pump. It can also be used in locations and emergency situations in which there is no external access to electricity.

There are already some self-contained head-worn positive airway pressure units in the prior art and on the market. These units are innovative and useful. However, energy efficiency and battery life remain challenging issues. Energy efficiency and battery life are much more critical for a self-contained head-worn unit than they are for a bedside air pump that is connected to an electrical wall outlet. Motors can use a lot of energy. The challenge is how to create a self-contained head-worn positive airway pressure unit that is very energy efficient so that a battery does not have to be continually recharged. An energy self-sufficient positive airway pressure unit would be ideal, but does not yet exist in the prior art.

The invention that is disclosed herein has the potential to provide a much more energy efficient form of positive airway pressure than is possible with the prior art. The invention that is disclosed herein can even offer the possibility of energy self-sufficiency by harvesting energy from a person's exhalation. In the extreme, this invention can offer the first self-contained head-worn positive airway pressure unit that is energy self-sufficient and never has to be recharged from an external power source.

There are millions people around the world with Obstructive Sleep Apnea (OSA) who are not receiving positive airway pressure treatment. Many of these people do not have access to dependable external power for direct operation of an air pump or for repeatedly recharging the battery thereof. For them, an energy-harvesting device that does not require an external source of power would be a tremendous breakthrough.

Also, even among people who have access to external power, many people cannot tolerate being tethered to a bedside unit by an air hose while they sleep. They get tangled up in the hose as they toss and turn during sleep. Also, rolling over onto the hose can cut off their air supply. Finally, an energy self-sufficient positive airway pressure option would be extremely useful for camping, for travel, and for emergency conditions (hurricanes, earthquakes, etc) during which external power is not available. The invention that is disclosed herein can meet these needs. This invention can provide a novel, unique, and advantageous positive airway pressure treatment option for the millions of people with Obstructive Sleep Apnea (OSA) who are not currently receiving positive airway pressure therapy.

### CATEGORIZATION AND REVIEW OF THE PRIOR ART

It can often be challenging to classify prior art into discrete categories. That is the case in this field. There are several hundred examples of potentially-relevant prior art related to this invention, ranging from prior art concerning harvesting energy from the human body to prior art that provides respiratory support for people with Obstructive Sleep Apnea (OSA) and other respiratory conditions.

However, classification of the prior art into categories, even if imperfect, can be an invaluable tool for reviewing the prior art, identifying its limitations, and setting the stage for discussion of the advantages of the present invention that is disclosed in subsequent sections. Towards this end, I have identified 18 general categories of prior art (including a final miscellaneous category), identified examples of prior art which appear to be best classified into these categories, and then identified key limitations of the prior art which should be addressed. These limitations are addressed by the invention which is disclosed in subsequent sections.

The 18 categories of prior art that I will now discuss are as follows: (1) energy harvested from muscle motion; (2) energy harvested from internal fluid flow; (3) energy harvested from internal biological source; (4) energy harvested from internal thermal energy; (5) energy harvested from external pressurized gas; (6) air pump/blower: wearable; (7) air pump/blower: portable, but not wearable; (8) air pump/blower: sensor interactive and variable pressure; (9) passive exhalation resistance device; (10) tongue engaging device: suction/friction; (11) tongue engaging device: implant/anchor; (12) tongue engaging device: nerve stimulation; (13) airway engaging device: stent or magnet; (14) outward force on body surface: external negative pressure; (15) mouth insert/appliance; (16) one-way valve in lung; (17) external response to sensor; and (18) other potentially-relevant art.

1. Energy Harvested from Muscle Motion

This first category of prior art includes methods, devices, and systems that harvest energy from the motion of human muscles. Some of these methods and devices harvest energy directly by connection to human muscles. Other devices in this category harvest energy indirectly, by connection with tissues of the human body that are, in turn, moved by human muscles. Most of this prior art transduces energy from movement of human muscles into electricity. This electricity is generally intended to power implanted medical devices, such as pacemakers, in order to eliminate the need for recharging a battery from an external power source.

For the purposes of this review, almost all of the examples of prior art that are included in this category are implanted devices that harvest energy from within the human body. A few external energy-harvesting devices (such as those attached to the surface of a human body) are included, but I have not included the wide range of external devices (hand cranks, shoe-based generators, etc.) that harvest energy from human motion.

Examples of methods and devices in the prior art that appear to involve energy harvested from muscle motion include the following: U.S. Pat. No. 3,456,134 (Ko 1969, “Piezoelectric Energy Converter for Electronic Implants”); U.S. Pat. No. 3,906,960 (Lehr 1975, “Medical Energy Converter”); U.S. Pat. No. 4,140,132 (Dahl 1979, “Variable Rate Timer for a Cardiac Pacemaker”); U.S. Pat. No. 4,245,640 (Hunt 1981, “Chest Motion Electricity Generating Device”); U.S. Pat. No. 4,690,143 (Schroeppel 1987, “Pacing Lead with Piezoelectric Power Generating Means”); U.S. Pat. No. 4,798,206 (Maddison et al. 1989, “Implanted Medical System Including a Self-Powered Sensing System”); U.S. Pat. No. 5,344,385 (Buck et al. 1994, “Step-Down Skeletal Muscle Energy Conversion System”); U.S. Pat. No. 5,431,694 (Snaper et al. 1995, “Bio-Operable Power Source”); U.S. Pat. No. 5,443,504 (Hill 1995, “Basic Skeletal Muscle Energy Conversion System”); U.S. Pat. No. 5,479,946 (Trumble 1996, “Muscle Energy Converter”); U.S. Pat. No. 5,540,729 (Weijand 1996, “Movement Powered Medical Pulse Generator Having a Full-Wave Rectifier with Dynamic Bias”); U.S. Pat. No. 5,653,676 (Buck et al. 1997, “Step-Down Skeletal Muscle Energy Conversion Method”); U.S. Pat. No. 5,701,919 (Buck et al. 1997, “Step-Down Skeletal Muscle Energy Conversion System”); U.S. Pat. No. 5,718,248 (Trumble et al. 1998, “Muscle Energy Converter Pump and Method of Pumping Fluid of a Patient”); U.S. Pat. No. 5,810,015 (Flaherty 1998, “Power Supply for Implantable Device”); U.S. Pat. No. 5,888,186 (Trumble et al. 1999, “Muscle Energy Converter Activated Assist System and Method”); U.S. Pat. No. 5,954,058 (Flaherty 1999, “Power Supply for Implantable Device”); U.S. Pat. No. 5,984,857 (Buck et al. 1999, “Step-Down Skeletal Muscle Energy Conversion System”); and U.S. Pat. No. 6,433,465 (McKnight et al. 2002, “Energy-Harvesting Device Using Electrostrictive Polymers”).

Examples of prior art in this category also include: U.S. Pat. No. 6,546,286 (Olson 2003, “Battery-Less, Human-Powered Electrotherapy Device”); U.S. Pat. No. 6,828,908 (Clark 2004, “Locator System with an Implanted Transponder Having an Organically-Rechargeable Battery”); U.S. Pat. No. 6,945,926 (Trumble 2005, “Muscle Energy Converter”); U.S. Pat. No. 7,203,551 (Houben et al. 2007, “Implantable Lead-Based Sensor Powered by Piezoelectric Transformer”); U.S. Pat. No. 7,345,407 (Tanner 2008, “Human Powered Piezoelectric Power Generating Device”); U.S. Pat. No. 7,414,351 (Ulm et al. 2008, “Energy Harvesting Device Manufactured by Print Forming Processes”); U.S. Pat. No. 7,715,918 (Melvin 2010, “Muscle Energy Converter with Smooth Continuous Tissue Interface”); U.S. Pat. No. 7,729,767 (Baker et al. 2010, “Implantable Generating System”); U.S. Pat. No. 7,729,768 (White et al. 2010, “Implantable Cardiac Motion Powered Piezoelectric Energy Source”); U.S. Pat. No. 7,800,278 (Ujihara et al. 2010, “Energy Harvesting By Means Of Thermo-Mechanical Device Utilizing Bistable Ferromagnets”); U.S. Pat. No. 7,902,727 (Sham et al. 2011, “Apparatus and Method for Generating Electricity Using Piezoelectric Material”); and U.S. Pat. No. 7,977,807 (Connor 2011, “Wearable Device to Generate Electricity from Human Movement”);

Examples of prior art in this category also include: U.S. Patent Applications 20040073267 (Holzer 2004, “Micro-Generator Implant”); 20040158294 (Thompson 2004, “Self-Powered Implantable Element”); 20090152990 (Brown et al. 2009, “Apparatus for In Vivo Energy Harvesting”); 20090216292 (Pless et al. 2009, “Devices, Methods, and Systems for Harvesting Energy in the Body”); 20100063557 (Imran 2010, “Energy Harvesting Mechanism”); 20100076517 (Imran 2010, “Energy Harvesting Mechanism for Medical Devices”); 20100114142 (Albrecht et al. 2010, “Powering Implantable Distension Systems Using Internal Energy Harvesting Means”); 20100171394 (Glenn et al. 2010, “Energy Harvesting for Implanted Medical Devices”); 20100298720 (Potkay 2010, “In Situ Energy Harvesting Systems for Implanted Medical Devices”); 20100317977 (Piaget et al. 2010, “Implantable Medical Device with Internal Piezoelectric Energy Harvesting”); 20100317978 (Maile et al. 2010, “Implantable Medical Device Housing Modified for Piezoelectric Energy Harvesting”); 20110208010 (McKenna 2011, “Motion Energy Harvesting with Wireless Sensors”); and 20110275947 (Feldman et al. 2011, “Cardiovascular Power Source for Automatic Implantable Cardioverter Defibrillators”).

2. Energy Harvested from Internal Fluid Flow

This category of prior art includes methods, devices, and systems that harvest energy from flowing fluid within the human body. Some of this prior art harvests energy directly from a flowing fluid. Other examples of art in this category harvest energy indirectly, from tissue movement caused by variation in fluid pressure such as pulsation of the walls of a blood vessel. Devices in the prior art in this category are generally implanted within the human body. They are generally intended to power an implantable medical device such as a pacemaker.

In some respects, this category could be viewed as a subset of the previous category concerning harvesting energy from the movement of human muscles. Fluid flow within the body can generally be traced back to muscle movement, especially the beating of the heart muscle. However, I have listed energy harvesting from fluid flow as a separate category because harvesting energy from fluid flow can be seen as being closer to harvesting energy from gas flow than to harvesting energy from movement of solid body members. Examples of methods and devices in the prior art that appear to involve harvesting energy from internal fluid flow include the following: U.S. Pat. No. 3,563,245 (McLean et al. 1971, “Biologically Implantable and Energized Power Supply”); U.S. Pat. No. 3,693,625 (Auphan 1972, “Heart Stimulator and Heart-Powered Energy Supply Therefor”); U.S. Pat. No. 3,943,936 (Rasor et al. 1976, “Self Powered Pacers And Stimulators”); U.S. Pat. No. 4,453,537 (Spitzer 1984, “Apparatus for Powering a Body Implant Device”); U.S. Pat. No. 6,822,343 (Estevez 2004, “Generating Electric Power in Response to Activity of a Biological System”); U.S. Pat. No. 6,827,682 (Bugge et al. 2004, “Implantable Device for Utilization of the Hydraulic Energy of the Heart”); U.S. Pat. No. 7,081,683 (Ariav 2006, “Method and Apparatus for Body Generation of Electrical Energy”); U.S. Pat. No. 7,560,856 (Chen et al. 2009, “Harvesting Energy from Flowing Fluid”); U.S. Pat. No. 7,813,810 (Cernasov 2010, “Apparatus and Method for Supplying Power to Subcutaneously Implanted Devices”); and U.S. Pat. No. RE41394 (Bugge et al. 2010, “Implantable Device for Utilization of the Hydraulic Energy of the Heart”); and U.S. Patent Application 20040021322 (Ariav 2004, “Method and Apparatus for Body Generation of Electrical Energy”).

3. Energy Harvested from Internal Biological Source

This category of prior art includes methods, devices, and systems that harvest energy from biological and/or chemical processes within the human body. This category includes implanted biological fuel cells and chemical fuel cells that generate electricity within the human body. Devices in this category are generally intended to power an implanted medical device such as a pacemaker. In an example, methods and devices in this category may use a person's own body tissue and biochemical processes to generate power. In other examples, methods and devices in this category may implant a biological fuel cell that contains foreign biological members or chemicals that are used to generate power.

Examples of methods and devices in the prior art that appear to involve harvesting energy from an internal biological source include the following: U.S. Pat. No. 3,305,399 (Davis 1967, “Microbial Process of Producing Electricity”); U.S. Pat. No. 3,421,512 (Frasier 1969, “Implanted Electrical Device with Biological Power Supply”); U.S. Pat. No. 3,774,243 (Ng et al. 1973, “Implantable Power System for an Artificial Heart”); U.S. Pat. No. 3,861,397 (Rao et al. 1975, “Implantable Fuel Cell”); U.S. Pat. No. 3,941,135 (von Sturm et al. 1976, “Pacemaker with Biofuel Cell”); U.S. Pat. No. 5,810,015 (Flaherty 1998, “Power Supply for Implantable Device”); U.S. Pat. No. 5,954,058 (Flaherty 1999, “Power Supply for Implantable Device”); U.S. Pat. No. 6,294,281 (Heller 2001, “Biological Fuel Cell and Method”); U.S. Pat. No. 6,500,571 (Liberatore et al. 2002, “Enzymatic Fuel Cell”); U.S. Pat. No. 6,503,648 (Wang 2003, “Implantable Fuel Cell”); U.S. Pat. No. 6,531,239 (Heller 2003, “Biological Fuel Cell and Methods”); U.S. Pat. No. 6,970,744 (Shelchuk 2005, “Bioenergy Generator”); U.S. Pat. No. 7,018,735 (Heller 2006, “Biological Fuel Cell and Methods”); U.S. Pat. No. 7,160,637 (Chiao et al. 2007, “Implantable, Miniaturized Microbial Fuel Cell”); U.S. Pat. No. 7,238,442 (Heller 2007, “Biological Fuel Cell and Methods”); U.S. Pat. No. 7,368,190 (Heller et al. 2008, “Miniature Biological Fuel Cell that is Operational Under Physiological Conditions, and Associated Devices and Methods”); U.S. Pat. No. 7,709,134 (Minteer et al. 2010, “Microfluidic Biofuel Cell”); U.S. Pat. No. 7,811,689 (Heller 2010, “Biological Fuel Cell and Methods”); U.S. Pat. No. 7,927,749 (Swift et al. 2011, “Microbial Fuel Cell and Method”); U.S. Pat. No. 7,976,968 (Siu et al. 2011, “Microbial Fuel Cell With Flexible Substrate and Micro-Pillar Structure”); U.S. Pat. No. 7,998,624 (Heller 2011, “Biological Fuel Cell and Methods”); U.S. Pat. No. 7,998,625 (Heller 2011, “Biological Fuel Cell and Methods”); and U.S. Pat. No. 8,048,547 (Ringeisen et al. 2011, “Biological Fuel Cells with Nanoporous Membranes”); and U.S. Patent Application 20050027332 (Avrahami et al. 2005, “Implanted Autonomic Energy Source”).

4. Energy Harvested from Internal Thermal Energy

This category of prior art includes methods and devices that harvest energy from thermal energy within the human body. Generally these devices use an energy differential to generate electricity. Devices in this category are generally intended to power implanted medical devices.

Examples of methods and devices in the prior art that appear to involve harvesting energy from internal thermal energy include the following: U.S. Pat. No. 6,131,581 (Leysieffer et al. 2000, “Process and Device for Supply of an at Least Partially Implanted Active Device with Electric Power”); U.S. Pat. No. 6,470,212 (Weijand et al. 2002, “Body Heat Powered Implantable Medical Device”); U.S. Pat. No. 6,640,137 (MacDonald 2003, “Biothermal Power Source for Implantable Devices”); U.S. Pat. No. 7,127,293 (MacDonald 2006, “Biothermal Power Source for Implantable Devices”); U.S. Pat. No. 7,340,304 (MacDonald 2008, “Biothermal Power Source for Implantable Devices”); U.S. Pat. No. 8,003,879 (Erbstoeszer et al. 2011, “Method and Apparatus for In Vivo Thermoelectric Power System”); and U.S. Pat. No. 8,039,727 (Erbstoeszer et al. 2011, “Method and Apparatus for Shunt for In Vivo Thermoelectric Power System”); and U.S. Patent Applications 20040093041 (MacDonald 2004, “Biothermal Power Source for Implantable Devices”); 20050171580 (MacDonald 2005, “Biothermal Power Source for Implantable Devices”); 20070251244 (Erbstoeszer et al. 2007, “Method and Apparatus for In Vivo Thermoelectric Power System”); 20070253227 (James et al. 2007, “Power Converter for Use with Implantable Thermoelectric Generator”); and 20100114142 (Albrecht et al. 2010, “Powering Implantable Distension Systems Using Internal Energy Harvesting Means”).

5. Energy Harvested from External Pressurized Gas

This category of prior art includes methods, devices, and systems that harvest energy from external pressurized gas that is outside the human body, such as a cylinder of compressed air or some other source of pressurized gas. In an example, a device in this category can use the force of pressurized air from a cylinder, or some other external source of pressurized gas, to power the operation of a blower for a mask. There are few devices in this category in the prior art. The few examples that were found appear to be primarily directed toward creating a portable ventilation system driven by a canister of pressurized gas.

Examples of methods and devices in the prior art that appear to be best classified in this category include the following: U.S. Pat. No. 5,553,454 (Mortner 1996, “Compressed Air Engine System and Method for Generating Electrical Energy from the Controlled Release of Compressed Air”); U.S. Pat. No. 5,969,429 (Rudolph et al. 1999, “Breathing Apparatus Having Electrical Power Supply Arrangement with Turbine-Generator Assembly”); and U.S. Pat. No. 7,218,009 (Hendrickson et al. 2007, “Devices, Systems and Methods for Generating Electricity from Gases Stored in Containers Under Pressure”); and U.S. Patent Applications 20100163046 (Fisher et al. 2010, “Method and Apparatus for Ventilation Assistance”); and 20100199985 (Hamilton et al. 2010, “Portable Gas Powered Positive Pressure Breathing Apparatus and Method”).

### 6. Air Pump/Blower: Wearable

This category of prior art includes methods, devices, and systems that feature an active energy-powered air-moving member (such as an electric air pump or blower) that is integrated into a member (such as a mask or vest) that is worn on the human body. Integration of an active energy-powered air-moving member into a mask, or other member that can be worn, helps to make a system of respiratory support more portable. Also, such a system does not require a tube connected to a separate air-moving member. For the purposes of this review, this category is viewed broadly. Masks have been included that serve various purposes, not just those that provide positive airway pressure for Obstructive Sleep Apnea (OSA). Masks have also been included that serve other functions such as air filtration (including gas masks), gas channeling, and respiratory ventilation.

Examples of methods and devices in the prior art that appear to include an active energy-powered air-moving member in a mask, or other member, that is worn on the human body include the following: U.S. Pat. No. 4,233,972 (Hauff et al. 1980, “Portable Air Filtering and Breathing Assist Device”); U.S. Pat. No. 4,549,542 (Chien 1985, “Multiple-Effect Respirator”); U.S. Pat. No. 4,886,056 (Simpson 1989, “Breathing Apparatus”); U.S. Pat. No. 4,944,310 (Sullivan 1990, “Device for Treating Snoring Sickness”); U.S. Pat. No. 5,035,239 (Edwards 1991, “Powered Respirators”); U.S. Pat. No. 5,303,701 (Heins et al. 1994, “Blower-Supported Gas Mask And Breathing Equipment With An Attachable Control Part”); U.S. Pat. No. 5,372,130 (Stern et al. 1994, “Face Mask Assembly and Method Having a Fan and Replaceable Filter”); U.S. Pat. No. 6,257,235 (Bowen 2001, “Face Mask with Fan Attachment”); U.S. Pat. No. 6,371,112 (Bibi 2002, “Device, System and Method for Preventing Collapse of the Upper Airway”); U.S. Pat. No. 6,435,184 (Ho 2002, “Gas Mask Structure”); U.S. Pat. No. 6,595,212 (Arnott 2003, “Method and Apparatus for Maintaining Airway Patency”); U.S. Pat. No. 6,629,529 (Arnott 2003, “Method for Maintaining Airway Patency”); U.S. Pat. No. 6,705,314 (O'Dea 2004, “Apparatus and Method for Relieving Dyspnoea”); U.S. Pat. No. 6,763,828 (Arnott 2004, “Apparatus for Maintaining Airway Patency”); U.S. Pat. No. 6,854,464 (Mukaiyama et al. 2005, “Respiration Protecting Apparatus”); U.S. Pat. No. 6,895,959 (Lukas 2005, “Gas Mask and Breathing Equipment with a Compressor”); U.S. Pat. No. 6,895,962 (Kullik et al. 2005, “Device for Supporting Respiration”); U.S. Pat. No. 7,195,014 (Hoffman 2007, “Portable Continuous Positive Airway Pressure System”); U.S. Pat. No. 7,195,015 (Kuriyama 2007, “Breathing Apparatus”); U.S. Pat. No. 7,464,705 (Tanizawa et al. 2008, “Powered Respirator”); U.S. Pat. No. 7,516,743 (Hoffman 2009, “Continuous Positive Airway Pressure Device and Configuration For Employing Same”); U.S. Pat. No. 7,823,590 (Bibi et al. 2010, “Devices for Preventing Collapse of the Upper Airway Methods for Use Thereof and Systems and Articles of Manufacture Including Same”); U.S. Pat. No. 7,874,290 (Chalvignac 2011, “Breathing Assistance Device”); U.S. Pat. No. 7,913,692 (Kwok 2011, “CPAP Mask and System”); U.S. Pat. No. 8,020,556 (Hayek 2011, “Respiratory Apparatus”); U.S. Pat. No. 8,020,557 (Bordewick et al. 2011, “Apparatus and Methods for Administration of Positive Airway Pressure Therapies”); and U.S. Pat. No. 8,069,853 (Tilley 2011, “Breath Responsive Powered Air-Purifying Respirator”).

Examples of prior art in this category also include: U.S. Patent Applications 20020104541 (Bibi et al. 2002, “Devices, Systems and Methods for Preventing Collapse of the Upper Airway and Sensors for Use Therein”); 20030066527 (Chen 2003, “Face Mask Having Device For Drawing Air Into The Mask”); 20030172930 (Kullik et al. 2003, “Device For Supporting Respiration”); 20040079373 (Mukaiyama et al. 2004, “Respiration Protecting Apparatus”); 20040168689 (Kuriyama 2004, “Respirator”); 20040216741 (Arnott 2004, “Apparatus for Maintaining Airway Patency”); 20040237965 (Bibi et al. 2004, “Devices for Preventing Collapse of the Upper Airway Methods for Use Thereof and Systems and Articles of Manufacture Including Same”); 20050034724 (O'Dea 2005, “Apparatus and Method for Relieving Dyspnoea”); 20060096596 (Occhialini et al. 2006, “Wearable System for Positive Airway Pressure Therapy”); 20060213516 (Hoffman 2006, “Portable Continuous Positive Airway Pressure System”); 20060237013 (Kwok 2006, “Ventilator Mask and System”); 20070000493 (Cox 2007, “Apparatus for Maintaining Airway Patency”); 20070246045 (Hoffman 2007, “Continuous Positive Airway Pressure Device and Configuration For Employing Same”); 20070251527 (Sleeper 2007, “Self-Contained Respiratory Therapy Apparatus for Enhanced Patient Compliance”); 20070277827 (Bordewick et al. 2007, “Apparatus and Methods for Administration of Positive Airway Pressure Therapies”); 20080029098 (Ottestad 2008, “Portable Breathing Apparatus”); 20080178879 (Roberts et al. 2008, “Impeller for a Wearable Positive Airway Pressure Device”); 20080216831 (McGinnis et al. 2008, “Standalone CPAP Device and Method of Using”); 20080216835 (McGinnis et al. 2008, “Standalone CPAP Device and Method of Using”); 20080251079 (Richey 2008, “Apparatus and Method for Providing Positive Airway Pressure”); 20100108070 (Kwok 2010, “Ventilator Mask and System”); 20100163043 (Hart et al. 2010, “Self-Contained Oral Ventilation Device”); 20100170513 (Bowditch et al. 2010, “Self-Contained, Intermittent Positive Airway Pressure Systems and Methods for Treating Sleep Apnea, Snoring, and Other Respiratory Disorders”); 20120000463 (Bordewick et al. 2012, “Apparatus and Methods for Administration of Positive Airway Pressure Therapies”); 20120152255 (Barlow et al. 2012, “PAP System”); 20120199124 (Bowditch et al. 2012, “Positive Airway Pressure System with Head Position Control”); 20120199125 (Bowditch et al. 2012, “Positive Airway Pressure System with Head Control”); 20120234323 (Connor 2012, “Energy-Harvesting Respiratory Method and Device”); and 20120266873 (Lalonde 2012, “Integrated Positive Airway Pressure Apparatus”).

### 7. Air Pump/Blower: Portable, but not Wearable

This category of prior art includes methods, devices, and systems that have an active energy-powered air-moving member (such as an air pump or blower) that is relatively portable, but wherein the air-moving member does not appear to be integrated into a mask, or other member, that is worn on the human body as was the case in the previous category. Methods and devices in this category generally include a separate air-moving member that is connected, via an air tube, to a mask or other member that is worn on the human body. For example, the air pump or blower may be a bedside unit. The boundary of this category is relatively imprecise because almost all positive airway pressure devices are portable to some extent. For the purposes of this review, we have included only those devices in the prior art that appear to be specifically designed to be portable with features such as: being battery-powered, being energy efficient, being light-weight, and/or being compact.

Examples of methods and devices in the prior art that appear to have an air pump or blower that is portable, but not wearable, include the following: U.S. Pat. No. 6,526,970 (DeVries et al. 2003, “Portable Drag Compressor Powered Mechanical Ventilator”); U.S. Pat. No. 6,877,511 (DeVries et al. 2005, “Portable Drag Compressor Powered Mechanical Ventilator”); U.S. Pat. No. 7,032,589 (Kerechanin et al. 2006, “Portable Ventilator”); U.S. Pat. No. 7,080,646 (Wiesmann et al. 2006, “Self-Contained Micromechanical Ventilator”); U.S. Pat. No. 7,188,621 (DeVries et al. 2007, “Portable Ventilator System”); U.S. Pat. No. 7,222,623 (DeVries et al. 2007, “Portable Drag Compressor Powered Mechanical Ventilator”); U.S. Pat. No. 7,320,321 (Pranger et al. 2008, “Self-Contained Micromechanical Ventilator”); U.S. Pat. No. 7,721,736 (Urias et al. 2010, “Self-Contained Micromechanical Ventilator”); U.S. Pat. No. 7,849,854 (DeVries et al. 2010, “Portable Drag Compressor Powered Mechanical Ventilator”); U.S. Pat. No. 7,866,944 (Kenyon et al. 2011, “Compact Low Noise Efficient Blower for CPAP Devices”); U.S. Pat. No. 7,942,380 (Bertinetti et al. 2011, “Portable Positive Airway Pressure Device Accessories and Methods for Use Thereof”); and U.S. Pat. No. 8,011,362 (Adams 2011, “Compact Continuous Positive Airway Pressure Apparatus and Method”); and U.S. Patent Applications 20080053438 (DeVries et al. 2008, “Portable Ventilator System”); 20080196720 (Kollmeyer et al. 2008, “Mobile Medical Ventilator”); 20100132708 (Martin et al. 2010, “Positive Airway Pressure Device”); 20100307487 (Dunsmore et al. 2010, “Respiratory Therapy Device and Method”); 20110203587 (Bertinetti et al. 2011, “Portable Positive Airway Pressure Device Accessories and Methods for Use Thereof”); 20110203592 (Adams 2011, “Compact Continuous Positive Airway Pressure Apparatus and Method”); and 20110214673 (Masionis 2011, “Portable Life Support Apparatus Ventilator”).

### 8. Air Pump/Blower: Sensor Interactive and Variable Pressure

This category of prior art includes methods, devices, and systems that use an active energy-powered air-moving member to provide respiratory support in an interactive and sophisticated manner that is based on a person's natural respiratory cycle or the occurrence (or prediction) of an adverse respiratory event. For example, positive airway pressure devices in this category can vary the amount of positive airway pressure over time, in an interactive manner, in synchronization with a person's natural breathing cycle. In other examples, a device in this category may increase the amount of pressure, in an interactive manner, in response to airway closure that is detected by a respiratory sensor or is predicted by an algorithm. This category is potentially very broad. For the purposes of this review, we have sought to include only those methods, devices, and systems in the prior art that appear to be most relevant to the present invention, such as those that provide Positive End Expiratory Pressure (PEEP) for Obstructive Sleep Apnea (OSA).

Examples of methods, devices, and systems in the prior art that appear to use an active energy-powered air-moving member to provide respiratory support in an interactive and sophisticated manner include the following: U.S. Pat. No. 4,506,666 (Durkan 1985, “Method and Apparatus for Rectifying Obstructive Apnea”); U.S. Pat. No. 4,823,788 (Smith et al. 1989, “Demand Oxygen Controller and Respiratory Monitor”); U.S. Pat. No. 5,134,995 (Gruenke et al. 1992, “Inspiratory Airway Pressure System with Admittance Determining Apparatus and Method”); U.S. Pat. No. 5,148,802 (Sanders et al. 1992, “Method and Apparatus for Maintaining Airway Patency to Treat Sleep Apnea and Other Disorders”); U.S. Pat. No. 5,199,424 (Sullivan et al. 1993, “Device for Monitoring Breathing During Sleep and Control of CPAP Treatment that is Patient Controlled”); U.S. Pat. No. 5,203,343 (Axe et al. 1993, “Method and Apparatus for Controlling Sleep Disorder Breathing”); U.S. Pat. No. 5,239,995 (Estes et al. 1993, “Sleep Apnea Treatment Apparatus”); U.S. Pat. No. 5,245,995 (Sullivan et al. 1993, “Device and Method for Monitoring Breathing During Sleep, Control of CPAP Treatment, and Preventing of Apnea”); U.S. Pat. No. 5,259,373 (Gruenke et al. 1993, “Inspiratory Airway Pressure System Controlled by the Detection and Analysis of Patient Airway Sounds”); U.S. Pat. No. 5,301,689 (Wennerholm 1994, “Device for Temporary Artificial Respiration Assistance for Persons Having Snore Problems”); U.S. Pat. No. 5,535,738 (Estes et al. 1996, “Method and Apparatus for Providing Proportional Positive Airway Pressure to Treat Sleep Disordered Breathing”); U.S. Pat. No. 5,645,054 (Cotner et al. 1997, “Device and Method for the Treatment of Sleep Apnea Syndrome”); U.S. Pat. No. 5,649,533 (Oren 1997, “Therapeutic Respiration Device”); U.S. Pat. No. 5,664,562 (Bourdon 1997, “Breathing Aid Device”); U.S. Pat. No. 5,845,636 (Gruenke et al. 1998, “Method and Apparatus for Maintaining Patient Airway Patency”); U.S. Pat. No. 5,868,133 (DeVries et al. 1999, “Portable Drag Compressor Powered Mechanical Ventilator”); U.S. Pat. No. 5,884,625 (Hart 1999, “Oral Appliance for Delivering Gas to the Retroglossal Area”); U.S. Pat. No. 5,918,597 (Jones et al. 1999, “PEEP Control in a Piston Ventilator”); U.S. Pat. No. 5,950,624 (Hart 1999, “Oral Appliance Having Hollow Body”); U.S. Pat. No. 5,953,713 (Behbehani et al. 1999, “Method and Apparatus for Treatment of Sleep Disorder Breathing Employing Artificial Neural Network”); and U.S. Pat. No. 6,085,747 (Axe et al. 2000, “Method and Apparatus for Controlling Sleep Disorder Breathing”). Examples of prior art in this category also include: U.S. Pat. No. 6,253,764 (Calluaud 2001, “Control of Delivery Pressure in CPAP Treatment or Assisted Respiration”); U.S. Pat. No. 6,283,119 (Bourdon 2001, “Breathing Aid Apparatus in Particular for Treating Sleep Apnoea”); U.S. Pat. No. 6,349,724 (Burton et al. 2002, “Dual-Pressure Blower for Positive Air Pressure Device”); U.S. Pat. No. 6,427,689 (Estes et al. 2002, “Sleep Apnea Treatment Apparatus”); U.S. Pat. No. 6,484,719 (Berthon-Jones 2002, “Method for Providing Ventilatory Assistance in a Spontaneously Breathing Subject”); U.S. Pat. No. 6,532,957 (Berthon-Jones 2003, “Assisted Ventilation to Match Patient Respiratory Need”); U.S. Pat. No. 6,629,527 (Estes et al. 2003, “Sleep Apnea Treatment Apparatus”); U.S. Pat. No. 6,810,876 (Berthon-Jones 2004, “Assisted Ventilation to Match Patient Respiratory Need”); U.S. Pat. No. 6,895,964 (McAuliffe et al. 2005, “Flow Diverter for Controlling the Pressure and Flow Rate in CPAP Device”); U.S. Pat. No. 6,948,497 (Zdrojkowski et al. 2005, “Breathing Gas Delivery Method and Apparatus”); U.S. Pat. No. 6,988,994 (Rapoport et al. 2006, “Positive Airway Pressure System and Method for Treatment of Sleeping Disorder in Patient”); U.S. Pat. No. 6,990,980 (Richey 2006, “Carbon Dioxide-Based Bi-Level CPAP Control”); U.S. Pat. No. 7,036,506 (McAuliffe et al. 2006, “Flow Diverter for Controlling the Pressure and Flow Rate in CPAP Device”); U.S. Pat. No. 7,044,129 (Truschel et al. 2006, “Pressure Support System and Method”); U.S. Pat. No. 7,100,607 (Zdrojkowski et al. 2006, “Breathing Gas Delivery Method and Apparatus”); U.S. Pat. No. 7,128,069 (Farrugia et al. 2006, “Method and Apparatus for Improving the Comfort of CPAP”); U.S. Pat. No. 7,152,598 (Morris et al. 2006, “System and Method for Providing a Breathing Gas”); U.S. Pat. No. 7,246,619 (Truschel et al. 2007, “Snore Detecting Method and Apparatus”); U.S. Pat. No. 7,284,554 (Shaw 2007, “Continuous Positive Airway Pressure Device”); U.S. Pat. No. 7,370,650 (Nadjafizadeh et al. 2008, “Gas Supply Device for Sleep Apnea”); and U.S. Pat. No. 7,448,383 (Delache et al. 2008, “Air Assistance Apparatus Providing Fast Rise And Fall Of Pressure Within One Patient”s Breath”).

Examples of prior art in this category further include: U.S. Pat. No. 7,469,697 (Lee et al. 2008, “Feedback System and Method for Sleep Disordered Breathing Therapy”); U.S. Pat. No. 7,527,055 (McAuliffe et al. 2009, “Flow Diverter for Controlling the Pressure and Flow Rate in CPAP Device”); U.S. Pat. No. 7,533,670 (Freitag et al. 2009, “Systems, Methods and Apparatus for Respiratory Support of a Patient”); U.S. Pat. No. 7,562,657 (Blanch et al. 2009, “Method And Apparatus For Non-Invasive Prediction of Intrinsic Positive End-Expiratory Pressure (PEEPi) in Patients Receiving Ventilator Support”); U.S. Pat. No. 7,575,005 (Mumford et al. 2009, “Mask Assembly with Integrated Sensors”); U.S. Pat. No. 7,694,679 (McAuliffe et al. 2010, “Flow Diverter for Controlling the Pressure and Flow Rate in CPAP Device”); U.S. Pat. No. 7,793,660 (Kimmel et al. 2010, “Method of Treating Obstructive Sleep Apnea”); U.S. Pat. No. 7,841,343 (Deane et al. 2010, “Systems and Methods for Delivering Therapeutic Gas to Patients”); U.S. Pat. No. 7,866,318 (Bassin 2011, “Methods For Providing Expiratory Pressure Relief In Positive Airway Pressure Therapy”); U.S. Pat. No. 7,901,361 (Rapoport et al. 2011, “Method and Apparatus for Optimizing the Continuous Positive Airway Pressure for Treating Obstructive Sleep Apnea”); U.S. Pat. No. 7,942,824 (Kayyali et al. 2011, “Integrated Sleep Diagnostic and Therapeutic System and Method”); U.S. Pat. No. 8,011,365 (Douglas et al. 2011, “Mechanical Ventilation in the Presence of Sleep Disordered Breathing”); U.S. Pat. No. 8,015,974 (Christopher et al. 2011, “System for Providing Flow-Targeted Ventilation Synchronized to a Patient's Breathing Cycle”); U.S. Pat. No. 8,020,558 (Christopher et al. 2011, “System for Providing Flow-Targeted Ventilation Synchronized to a Patient's Breathing Cycle”); U.S. Pat. No. 8,051,853 (Berthon-Jones 2011, “Method and Apparatus for Providing Ventilatory Assistance”); U.S. Pat. No. 8,068,904 (Sun et al. 2011, “Devices and Methods for Monitoring Physiological Information Relating to Sleep with an Implantable Device”); and 8215302 (Kassatly et al. 2012, “Discontinuous Positive Airway Pressure Device and Method of Reducing Sleep Disordered Breathing Events”).

Examples of prior art in this category further include: U.S. Patent Applications 20070215156 (Kwok 2007, “Snoring Treatment Apparatus and Methods of Managing Snorers”); 20080142013 (Hallett et al. 2008, “Exhaust Apparatus for Use in Administering Positive Pressure Therapy Through the Nose or Mouth”); 20090020121 (Bassin 2009, “Methods for Providing Expiratory Pressure Relief in Positive Airway Pressure Therapy”); 20100180895 (Kwok et al. 2010, “Methods and Apparatus for Controlling Mask Leak in CPAP Treatment”); 20100252042 (Kapust et al. 2010, “Methods, Systems and Devices for Non-Invasive Open Ventilation for Treating Airway Obstructions”); 20100269834 (Freitag et al. 2010, “Systems, Methods and Apparatus for Respiratory Support of a Patient”); 20100313898 (Richard et al. 2010, “Apparatus and Methods for Treating Sleep Related Disorders”); 20110073110 (Kenyon et al. 2011, “Compact Low Noise Efficient Blower for CPAP Devices”); 20110079224 (Arnott 2011, “System, Apparatus and Method for Maintaining Airway Patency and Pressure Support Ventilation”); 20110284003 (Douglas et al. 2011, “Mechanical Ventilation in the Presence of Sleep Disordered Breathing”); 20110295083 (Doelling et al. 2011, “Devices, Systems, and Methods for Monitoring, Analyzing, and/or Adjusting Sleep Conditions”); 20120111331 (Witt et al. 2012, “System and Respiration Appliance for Supporting the Airway of a Subject”); 20120266887 (Geoffrey et al. 2012, “Double-Ended Blower and Volutes Therefor”); 20120291783 (Peiris et al. 2012, “Breathing Assistance Apparatus”); and 20120291785 (Ramanan et al. 2012, “Methods and Apparatus for Adaptable Pressure Treatment of Sleep Disordered Breathing”).

### 9. Passive Exhalation Resistance Device

This category of prior art includes methods, devices, and systems that provide passive resistance to exhalation. They do not use an active energy-powered air-moving member such as an air pump or blower. Passive resistance to exhalation can be used to provide Positive End Expiratory Pressure (PEEP) for respiratory conditions such as Obstructive Sleep Apnea (OSA). This category includes devices that are integrated into a member (such as a mask, nasal insert, or mouth appliance) that is attached to the human body and/or covers a person's nasal and/or oral openings. Some such devices have an airflow valve that offers greater resistance to gas outflow, during exhalation, and less resistance to gas inflow, during inhalation.

Devices in this category are more portable than devices to provide positive airway pressure that require a connection to an external power source. Over time, devices in this category are also more portable than battery-powered positive airway pressure devices because the batteries of the latter devices must be repeatedly recharged. Other potential advantages of devices in this category include their simplicity of operation, reduced noise compared to energy-powered blowers, and the freedom of movement that they offer to people who would otherwise be tethered to an air tube while sleeping. One could argue that handheld devices that can be held against a person's face in order to cover nasal and/or oral openings should not be included here because they are not helpful for providing respiratory support while a person sleeps. However, in this review, handheld devices are included in this category for the sake of completeness.

Examples of methods and devices in the prior art that appear to provide respiratory support through passive resistance to exhalation include the following: U.S. Pat. No. 7,468,69 (Moulton 1903, “Device for Preventing Snoring”); U.S. Pat. No. 3,908,987 (Boehringer 1975, “Controlled Positive End Pressure Expiratory Device”); U.S. Pat. No. 5,018,517 (Liardet 1991, “Expiration-Resisting Apparatus Designed for Improving Pulmonary Ventilation”); U.S. Pat. No. 5,658,221 (Hougen 1997, “Portable Personal Breathing Apparatus and Method of Using Same”); U.S. Pat. No. 5,890,998 (Hougen 1999, “Portable Personal Breathing Apparatus”); U.S. Pat. No. 6,425,393 (Lurie et al. 2002, “Automatic Variable Positive Expiratory Pressure Valve And Methods”); U.S. Pat. No. 6,510,846 (O'Rourke 2003, “Sealed Back Pressure Breathing Device”); U.S. Pat. No. 6,581,598 (Foran et al. 2003, “Positive Expiratory Pressure Device”); U.S. Pat. No. 6,659,100 (O'Rourke 2003, “Sealed Back Pressure Breathing Device”); U.S. Pat. No. 6,722,360 (Doshi 2004, “Methods and Devices for Improving Breathing in Patients with Pulmonary Disease”); U.S. Pat. No. 6,786,216 (O'Rourke 2004, “Sealed Back Pressure Breathing Device”); U.S. Pat. No. 6,883,518 (Mittelstadt et al. 2005, “Unidirectional Respirator Valve”); U.S. Pat. No. 6,997,177 (Wood 2006, “Ventilation Interface for Sleep Apnea Therapy”); U.S. Pat. No. 7,059,324 (Pelerossi et al. 2006, “Positive Expiratory Pressure Device with Bypass”); U.S. Pat. No. 7,334,581 (Doshi 2008, “Methods and Devices for Improving Breathing in Patients with Pulmonary Disease”); U.S. Pat. No. 7,506,649 (Doshi et al. 2009, “Nasal Devices”); U.S. Pat. No. 7,699,054 (Pelerossi et al. 2010, “Positive Expiratory Pressure Device”); U.S. Pat. No. 7,735,491 (Doshi et al. 2010, “Methods of Treating Respiratory Disorders”); U.S. Pat. No. 7,735,492 (Doshi et al. 2010, “Nasal Respiratory Devices”); U.S. Pat. No. 7,779,841 (Dunsmore et al. 2010, “Respiratory Therapy Device and Method”); U.S. Pat. No. 7,798,148 (Doshi et al. 2010, “Respiratory Devices”); U.S. Pat. No. 7,806,120 (Loomas et al. 2010, “Nasal Respiratory Devices for Positive End-Expiratory Pressure”); U.S. Pat. No. 7,856,979 (Doshi et al. 2010, “Nasal Respiratory Devices”); U.S. Pat. No. 7,987,852 (Doshi et al. 2011, “Nasal Devices”); U.S. Pat. No. 7,992,563 (Doshi 2011, “Methods and Devices for Improving Breathing in Patients with Pulmonary Disease”); U.S. Pat. No. 7,992,564 (Doshi et al. 2011, “Respiratory Devices”); U.S. Pat. No. 8,020,700 (Doshi et al. 2011, “Packaging and Dispensing Nasal Devices”); U.S. Pat. No. 8,025,054 (Dunsmore et al. 2011, “Passive Respiratory Therapy Device”); U.S. Pat. No. 8,061,357 (Pierce et al. 2011, “Adhesive Nasal Respiratory Devices”); and U.S. Pat. No. 8,240,309 (Doshi et al. 2012 “Adjustable Nasal Devices”).

Examples of prior art in this category also include: U.S. Patent Applications 20060144398 (Doshi et al. 2006, “Respiratory Devices”); 20060150978 (Doshi et al. 2006, “Methods of Treating Respiratory Disorders”); 20060150979 (Doshi et al. 2006, “Nasal Respiratory Devices”); 20070277832 (Doshi et al. 2007, “Nasal Respiratory Devices”); 20070283962 (Doshi et al. 2007, “Layered Nasal Devices”); 20070295338 (Loomas et al. 2007, “Nasal Respiratory Devices For Positive End-Expiratory Pressure”); 20080041373 (Doshi et al. 2008, “Nasal Devices”); 20080173309 (Doshi 2008, “Methods and Devices for Improving Breathing in Patients with Pulmonary Disease”); 20080178874 (Doshi et al. 2008, “Adjustable Nasal Devices”); 20090050144 (Pierce et al. 2009, “Adhesive Nasal Respiratory Devices”); 20090145441 (Doshi et al. 2009, “Delayed Resistance Nasal Devices and Methods of Use”); 20090188493 (Doshi et al. 2009, “Nasal Devices”); 20090194100 (Minagi 2009, “Nostril Plug for Improving Articulatory Disorder”); 20090194109 (Doshi et al. 2009, “CPAP Interface and Backup Devices”); 20090241965 (Sather et al. 2009, “Nasal Devices with Noise-Reduction and Methods of Use”); 20090308398 (Ferdinand et al. 2009, “Adjustable Resistance Nasal Devices”); 20100326447 (Loomas et al. 2010, “Nasal Respiratory Devices for Positive End-Expiratory Pressure”); 20100331877 (Li et al. 2010, “Airflow Restriction System”); 20110005520 (Doshi et al. 2011, “Quiet Nasal Respiratory Devices”); 20110005529 (Doshi et al. 2011, “Methods of Treating a Sleeping Subject”); 20110005530 (Doshi et al. 2011, “Methods of Treating a Disorder by Inhibiting Expiration”); 20110056499 (Doshi et al. 2011, “Sealing Nasal Devices for Use While Sleeping”); 20110067708 (Doshi et al. 2011, “Nasal Devices for Use While Sleeping”); 20110067709 (Doshi et al. 2011, “Nasal Respiratory Devices”); 20110203598 (Favet et al. 2011, “Nasal Devices Including Layered Nasal Devices and Delayed Resistance Adapters for Use with Nasal Devices”); 20110218451 (Lai et al. 2011, “Nasal Devices, Systems and Methods”); 20110220123 (Robson 2011, “Anti-Snoring Device Using Naturally Generated Positive Pressure”); 20110240038 (Doshi et al. 2011, “Nasal Devices”); and 20110290256 (Sather et al. 2011, “Layered Nasal Respiratory Devices”).

### 10. Tongue Engaging Device: Suction/Friction

This category of prior art includes methods, devices, and systems that engage the exterior surface of the tongue in order to draw it forward and keep it from blocking the airway. Such devices can be useful when the tongue would otherwise slide backwards and block the airway during sleep. This can be one cause of Obstructive Sleep Apena (OSA). Some of the methods and devices in this category involve engaging the exterior of the tongue using suction. For example, some devices are mouth appliances with suction tubes that engage the tongue. Other methods and devices in this category involve engaging the exterior of the tongue through other means such as clamps, elastic bands, or even peristaltic motion.

Examples of methods and devices in the prior art that appear to engage the exterior of the tongue to draw it forward and keep it out of the airway include the following: U.S. Pat. No. 5,957,133 (Hart 1999, “Oral Appliance with Negative Air Supply for Reducing Sleep Apnea and Snoring”); U.S. Pat. No. 6,055,986 (Meade 2000, “Apparatus and Method for the Reduction of Snoring”); U.S. Pat. No. 6,494,209 (Kulick 2002, “Method and Apparatus for Treatment of Snoring, Hypopnea and Apnea”); U.S. Pat. No. 6,877,513 (Scarberry et al. 2005, “Intraoral Apparatus for Enhancing Airway Patency”); U.S. Pat. No. 7,954,494 (Connor 2011, “Device with Actively-Moving Members that Hold or Move the Tongue”); U.S. Pat. No. 8,028,705 (Li 2011, “Tongue Retention System”); and U.S. Pat. No. 8,074,656 (Vaska et al. 2011, “Methods and Systems for Creating Pressure Gradients to Improve Airway Patency”); and U.S. Patent Applications 20090120446 (Vaska et al. 2009, “Methods and Systems for Improving Airway Patency”); 20100139668 (Harrington 2010, “Method and Device for Treatment of Obstructive Sleep Apnea”); 20110073119 (Chen et al. 2011, “Negative Pressure Oral Apparatus”); 20110180075 (Chen et al. 2011, “Adjustable Oral Interface and Method to Maintain Upper Airway Patency”); 20110180076 (Hegde et al. 2011, “Wearable Tissue Retention Device”); 20110192404 (Chen 2011, “Automated Negative Pressure Oral Apparatus”); 20110220124 (Vaska et al. 2011, “Methods and Systems for Improving Airway Patency”); and 20110259346 (Tsuiki et al. 2011, “Tongue Position Controller”).

### 11. Tongue Engaging Device: Implant/Anchor

This category of prior art includes methods, devices, and systems that involve implanting tongue-restraining or tongue-moving members inside the tongue in order to keep it, or move it, forward and out of the airway. Such implants can useful when the tongue would otherwise slide backwards and block the airway during sleep in Obstructive Sleep Apnea (OSA). Some of these methods and devices involve implantation of a tissue anchor in the posterior portion of the tongue and then connecting this anchor to an anterior structure such as the jaw bone or a dental appliance. The tissue anchor pulls the tongue forward. Other methods and devices in this category involve implantation of magnets in the tongue. These magnets, when engaged by a magnetic field, move the tongue forward by magnetic attraction or repulsion.

Examples of methods and devices in the prior art that appear to involve implants within the tongue to pull the tongue forward include the following: U.S. Pat. No. 7,644,714 (Atkinson et al. 2010, “Devices and Methods for Treating Sleep Disorders”); U.S. Pat. No. 7,658,192 (Harrington 2010, “Method and Device for Treatment of Obstructive Sleep Apnea”); U.S. Pat. No. 7,909,038 (Hegde et al. 2011, “Tongue Stabilization Device and Methods of Using the Same”); U.S. Pat. No. 7,921,850 (Nelson et al. 2011, “Systems and Methods for Moving and/or Restraining Tissue in the Upper Respiratory System”); U.S. Pat. No. 7,934,506 (Woodson et al. 2011, “System and Method for Temporary Tongue Suspension”); U.S. Pat. No. 7,975,700 (Frazier et al. 2011, “System for Adjustable Tissue Anchors”); U.S. Pat. No. 8,047,206 (Boucher et al. 2011, “Magnetic Devices, Systems, and Methods Placed In or On a Tongue”); and U.S. Pat. No. 8,074,655 (Sanders 2011, “Methods and Devices for Treating Sleep Apnea and Snoring”); and U.S. Patent Applications 20080060660 (Nelson et al. 2008, “Systems and Methods for Moving and/or Restraining Tissue in the Upper Respiratory System”); 20100132719 (Jacobs et al. 2010, “Implant Systems and Methods for Treating Obstructive Sleep Apnea”); 20100137905 (Weadock et al. 2010, “Implant Systems and Methods for Treating Obstructive Sleep Apnea”); 20110166598 (Gonazles et al. 2011, “Devices and Methods for Tongue Stabilization”); 20110308529 (Gillis et al. 2011, “Systems and Methods for Treatment of Sleep Apnea”); and 20110308530 (Gillis et al. 2011, “Systems and Methods for Treatment of Sleep Apnea”).

### 12. Tongue Engaging Device: Nerve Stimulation

This category of prior art includes methods, devices, and systems that use electrical impulses to stimulate nerves that innervate the muscles that control movement of the tongue or other soft tissue along the airway. Stimulation of these nerves causes the tongue, or other soft tissue, to move out of the airway. For example, some methods and devices in this category involve stimulation of the HypoGlossal Nerve (HGN) that controls the tongue and soft palate muscles. Generally, although not always, the device that provides nerve stimulation is implanted within the body in a manner similar to the way in which a pacemaker is implanted. This can be a useful approach for treating Obstructive Sleep Apnea (OSA).

Examples of methods and devices in the prior art that appear to involve stimulation of nerves to move the tongue or other soft tissue out of the airway include include the following: U.S. Pat. No. 5,123,425 (Shannon et al. 1992, “Obstructive Sleep Apnea Collar”); U.S. Pat. No. 7,025,730 (Cho et al. 2006, “System and Method for Automatically Monitoring and Delivering Therapy for Sleep-Related Disordered Breathing”); U.S. Pat. No. 7,809,442 (Bolea et al. 2010, “Obstructive Sleep Apnea Treatment Devices, Systems and Methods”); U.S. Pat. No. 7,937,159 (Lima et al. 2011, “Apparatus, System and Method for Therapeutic Treatment of Obstructive Sleep Apnea”); and 8024044 (Kirby et al. 2011, “Method and Apparatus for Hypoglossal Nerve Stimulation”); and U.S. Patent Applications 20070173893 (Pitts 2007, “Method and Apparatus for Preventing Obstructive Sleep Apnea”); 20080109047 (Pless 2008, “Apnea Treatment Device”); 20100121406 (Libbus et al. 2010, “Neural Stimulator to Treat Sleep Disordered Breathing”); 20100198306 (Lima et al. 2010, “Apparatus, System and Method for Therapeutic Treatment of Obstructive Sleep Apnea”); 20110071591 (Bolea et al. 2011, “Obstructive Sleep Apnea Treatment Devices, Systems and Methods”); 20110112601 (Meadows et al. 2011, “System for Stimulating a Hypoglossal Nerve for Controlling the Position of a Patient's Tongue”); 20110152966 (Bolea et al. 2011, “Obstructive Sleep Apnea Treatment Devices, Systems and Methods”); 20110196445 (Bolea et al. 2011, “Obstructive Sleep Apnea Treatment Devices, Systems and Methods”); 20110202106 (Bolea et al. 2011, “Obstructive Sleep Apnea Treatment Devices, Systems and Methods”); 20110264164 (Christopherson et al. 2011, “Method of Treating Sleep Disordered Breathing”); and 20110301679 (Rezai et al. 2011, “Apparatus and Method for Treating Pulmonary Conditions”).

### 13. Airway Engaging Device: Stent or Magnet

This category of prior art includes methods and devices that involve implanting a tissue-supporting scaffold (such as a stent) or some other implant in the airway in order to physically prop the airway open. One example of devices in this category are stents that are implanted in the tissue surrounding the airway to prop the airway open regardless of the pressure level in the airway. Another example of devices in this category are magnets that are implanted in the tissue surrounding the airway methods. In the case of magnets, electromagnetic repulsion or attraction provides the force to keep the airway open.

Examples of methods and devices in the prior art that appear to involve implantation of stents, magnets, or other members in airway tissue to keep the airway open include the following: U.S. Pat. No. 7,958,895 (Nelson et al. 2011, “Magnetic Force Devices, Systems, and Methods for Resisting Tissue Collapse within the Pharyngeal Conduit”); U.S. Pat. No. 7,958,896 (Nelson et al. 2011, “Magnetic Force Devices, Systems, and Methods for Resisting Tissue Collapse within the Pharyngeal Conduit”); U.S. Pat. No. 7,992,566 (Pflueger et al. 2011, “Apparatus and Methods for Treating Sleep Apnea”) and U.S. Pat. No. 7,997,266 (Frazier et al. 2011, “System and Method for Airway Manipulation”); and U.S. Patent Applications 20100280626 (Shalon et al. 2010, “Devices and Methods for Treating Sleep Disordered Breathing”); 20100319711 (Hegde et al. 2010, “Airway Implant and Methods of Making and Using”); and 20110290258 (Pflueger et al. 2011, “Apparatus and Methods for Treating Sleep Apnea”).

### 14. Outward Force on Body Surface

This category of prior art includes methods, devices, and system that seek to keep the airway open by applying outward force on neck tissue or some other portion of the body surface. The intent is to pull soft tissue near the body's exterior outward from the core of the body, which pulls soft tissue near the airway outward, which keeps the airway open. Some methods and devices in this category use negative pressure (such as suction) to engage the exterior surface of the neck, or some other portion of the body exterior, and pull it outwards. Other methods and devices in this category use adhesion to engage the exterior surface of the neck, or some other portion of the body, and draw it outwards. There are relatively few examples of this approach in the prior art. Examples of methods and devices in the prior art that appear to seek to treat respiratory conditions by exerting outward force on a body surface including the following: U.S. Pat. No. 7,762,263 (Aarestad et al. 2010, “Device and Method for Opening an Airway”) and U.S. Pat. No. 7,793,661 (Macken 2010, “Method and Apparatus for Treatment of Snoring and Sleep Apnea”); and U.S. Patent Applications 20030167018 (Wyckoff 2003, “Sleep Apnea Device and Method Thereof”); 20100275910 (Aarestad et al. 2010, “Device and Method for Opening an Airway”); and 20110066086 (Aarestad et al. 2011, “Device and Method for Opening an Airway”).

### 15. Mouth Insert/Appliance

This category of prior art includes methods, devices, and systems that are inserted into the mouth in order to address a respiratory condition such as Obstructive Sleep Apnea (OSA) or snoring. Some of the devices in this category engage the teeth to move the jaw forward. Moving the jaw forward is intended to move the tongue, or other soft tissue, forward and away from the airway. Other methods and devices in this category seek to address respiratory conditions by changing the air pressure in the oral cavity. Other methods and devices in this category are simply designed to keep the mouth closed and prevent airflow through the mouth. There are a few examples of advanced mouth inserts that include an air pump or blower that is integrated into a mouth insert that a person wears. Advanced devices with an active energy-using air moving member that are worn have been included in the “Air Pump/Blower: Wearable” category above (category 6) because such integration is their dominant feature with respect to this review of the prior art.

Examples of methods and devices in the prior art that appear to include a mouth insert or mouth appliance to treat a respiratory condition, without an integrated active air-moving member, include the following: U.S. Pat. No. 5,678,567 (Thornton et al. 1997, “Method and Apparatus for Adjusting a Dental Device”); U.S. Pat. No. 5,826,579 (Remmers et al. 1998, “Remote-Controlled Mandibular Positioning Device and Method of Using the Device”); U.S. Pat. No. 5,921,942 (Remmers et al. 1999, “Adaptively Controlled Mandibular Positioning Device and Method of Using the Device”); U.S. Pat. No. 5,954,048 (Thornton 1999, “Device and Method for Improving Breathing”); U.S. Pat. No. 5,983,892 (Thornton 1999, “Device for Improving Breathing”); U.S. Pat. No. 6,155,262 (Thornton et al. 2000, “Method and Apparatus for Adjusting a Dental Device”); U.S. Pat. No. 6,273,859 (Remmers et al. 2001, “Adaptively Controlled Mandibular Positioning Device and Method of Using the Device”); U.S. Pat. No. 6,305,376 (Thornton 2001, “Device and Method for Improving Breathing”); U.S. Pat. No. 6,374,824 (Thornton 2002, “Device for Improving Breathing”); U.S. Pat. No. 6,405,729 (Thornton 2002, “Oral Appliance for Improving Breathing and Method of Constructing Same”); U.S. Pat. No. 6,845,774 (Gaskell 2005, “Dental Device”); and 7650885 (Paoluccio et al. 2010, “Mouthpiece and Mask for Ventilation Assistance and Connector for Joining Objects”); and U.S. Patent Applications 20050081859 (Scarberry et al. 2005, “Intraoral Apparatus for Enhancing Airway Patency”); 20050236003 (Meader 2005, “Apnea Nipple and Oral Airway and Mandibular Advancement Device”); 20090078273 (Bhat et al. 2009, “Smart Mandibular Repositioning System”); and 20110232652 (Levendowski et al. 2011, “Systems and Methods for Optimizing Oral Appliance Therapy for the Treatment of Sleep Apnea”).

### 16. One-Way Valve in Lung

This category of prior art includes methods, devices, and systems that involve implanting a one-way valve (partial or complete) into an air passage within a lung. Although counter-intuitive in some respects, this approach can help in the treatment of Chronic Obstructive Pulmonary Disease (COPD). The valve serves to isolate a diseased portion of the lung from the good portions of the lung. This can prevent the disease from spreading from the bad sections of the lung to the good sections of the lung. To date, implantation of a one-way valve somewhere along the interior airway does not appear to have been proposed to treat Obstructive Sleep Apnea (OSA), but we include this category in this review for the sake of completeness.

Examples of methods and devices in the prior art that appear to involve implanting a one-way valve into a lung (or elsewhere in the central airway) include the following: U.S. Pat. No. 7,406,963 (Chang et al. 2008, “Variable Resistance Pulmonary Ventilation Bypass Valve and Method”); U.S. Pat. No. 7,686,013 (Chang et al. 2010, “Variable Resistance Pulmonary Ventilation Bypass Valve”); U.S. Pat. No. 7,726,305 (Chang et al. 2010, “Variable Resistance Pulmonary Ventilation Bypass Valve”) and U.S. Pat. No. 7,875,048 (Dillard et al. 2011, “One-Way Valve Devices for Anchored Implantation in a Lung”).

### 17. External Response to Sensor

This category of prior art includes methods, devices, and systems that involve external (generally non-therapeutic) responses, such as alarms, to respiratory events. There are exceptions, but these responses are generally not directly therapeutic in themselves. These responses are generally intended to provoke a therapeutic response on the part of a person who hears an alarm. Some methods and devices in this category involve an alarm that sounds in response to an adverse respiratory event. Other methods and devices in this category involve prompting a change in body position in response to an adverse respiratory event. In the latter case, the hope is that prompting a change in body position will help to correct the adverse respiratory event.

Examples of methods and devices in the prior art that appear to involve external (generally non-therapeutic) responses to respiratory events include the following: U.S. Pat. No. 6,371,120 (Chiu et al. 2002, “Snore Elimination Device”); U.S. Pat. No. 6,386,201 (Fard 2002, “Apparatus for Preventing Snoring”); U.S. Pat. No. 7,387,608 (Dunlop et al. 2008, “Apparatus and Method for the Treatment of Sleep Related Disorders”); U.S. Pat. No. 7,716,988 (Ariav et al. 2010, “Apparatus for Use in Controlling Snoring and Sensor Unit Particularly Useful Therein”); U.S. Pat. No. 7,725,195 (Lima et al. 2010, “RFID-Based Apparatus, System, and Method for Therapeutic Treatment of Obstructive Sleep Apnea”); U.S. Pat. No. 7,789,837 (Lehrman et al. 2010, “System and Method for Treating Obstructive Sleep Apnea”); and U.S. Pat. No. 7,866,212 (Ariav et al. 2011, “High-Sensitivity Sensors for Sensing Various Physiological Phenomena, Particularly Useful in Anti-Snoring Apparatus and Methods”); and U.S. Patent Applications 20080221470 (Sather et al. 2008, “Respiratory Sensor Adapters for Nasal Devices”) and 20100078017 (Andrieux et al. 2010, “Wireless Communications for a Breathing Assistance System”).

### 18. Other Potentially-Relevant Art

This last category of prior art is a miscellaneous category. This category includes a variety of methods, devices, and systems related to energy harvesting and/or providing respiratory support that are not easy to classify, but may nonetheless be relevant to the present invention. Examples of methods, devices, and systems in the prior art that have been included in this miscellaneous category are as follows: U.S. Pat. No. 3,268,845 (Whitmore 1966, “Respiration and Movement Transducer”); U.S. Pat. No. 3,837,337 (LaViolette 1974, “Self-Contained Closed Circuit Breathing Apparatus”); U.S. Pat. No. 4,821,712 (Gossett 1989, “Breathing Apparatus”); U.S. Pat. No. 5,048,517 (Pasternack 1991, “Recirculating Positive-Pressure Respirator”); U.S. Pat. No. 5,687,715 (Landis et al. 1997, “Nasal Positive Airway Pressure Apparatus and Method”); U.S. Pat. No. 5,810,015 (Flaherty 1998, “Power Supply for Implantable Device”); U.S. Pat. No. 6,302,105 (Wickham et al. 2001, “Apparatus for Supplying Breathable Gas”); U.S. Pat. No. 6,401,714 (Giorgini 2002, “Self Contained Breathing Apparatus”); U.S. Pat. No. 6,411,852 (Danek et al. 2002, “Modification of Airways by Application of Energy”); U.S. Pat. No. 6,457,471 (Bibi 2002, “Dual-Purpose Medical Device for Upper Airway Treatment and Methods for Using Same”); U.S. Pat. No. 6,772,762 (Piesinger 2004, “Personal Powered Air Filtration, Sterilization, and Conditioning System”); U.S. Pat. No. 6,792,942 (Ho et al. 2004, “Sleep Silencer”); U.S. Pat. No. 7,066,177 (Pittaway et al. 2006, “Exhalation Valves”); U.S. Pat. No. 7,080,645 (Genger et al. 2006, “Anti-Snoring Device, Method for Reducing Snoring, and a Nasal Air Cannula”); U.S. Pat. No. 7,114,497 (Aylsworth et al. 2006, “Method and System of Individually Controlling Airway Pressure of a Patient's Nares”); U.S. Pat. No. 7,275,542 (Lurie et al. 2007, “Bag-Valve Resuscitation for Treatment of Hypotension, Head Trauma, and Cardiac Arrest”); U.S. Pat. No. 7,406,966 (Wondka 2008, “Method and Device for Non-Invasive Ventilation with Nasal Interface”); U.S. Pat. No. 7,451,766 (Miller 2008, “Enhanced Breathing Device”); U.S. Pat. No. 7,562,659 (Matarasso 2009, “Respiratory Aid Apparatus and Method”); U.S. Pat. No. 7,835,529 (Hernandez et al. 2010, “Sound Canceling Systems and Methods”); U.S. Pat. No. 7,909,035 (Thornton 2011, “Multi-Chamber Mask and Method of Forming the Same”); U.S. Pat. No. 7,951,357 (Gross et al. 2011, “Implantable Power Sources and Sensors”); U.S. Pat. No. 7,967,014 (Heidmann et al. 2011, “Application Device for Breathing Mask Arrangement”); U.S. Pat. No. 8,037,885 (Metzger et al. 2011, “Treatment for Sleep Apnea or Snoring”); U.S. Pat. No. 8,051,850 (Kwok et al. 2011, “Nasal Dilator”); and U.S. Pat. No. 8,074,647 (Truitt et al. 2011, “Impeller and a Pressure Support System and Method Using Such a Method”).

Examples of prior art in this miscellaneous category also include: U.S. Patent Applications 20060180149 (Matarasso 2006, “A Respiratory Aid System and Method”); 20080060649 (Veliss et al. 2008, “Delivery of Respiratory Therapy”); 20080135044 (Freitag et al. 2008, “Methods and

Devices for Minimally Invasive Respiratory Support”); 20080142018 (Doshi et al. 2008, “Nasal Device Applicators”); 20090165799 (Duquette et al. 2009, “Continuous Positive Airway Pressure Device”); 20090320842 (Doherty et al. 2009, “Mask and Flow Generator System”); 20100000543 (Berthon-Jones et al. 2010, “Mask and Components Thereof”); 20100006097 (Frater et al. 2010, “Quiet Blower Apparatus and System and Method for Reducing Blower Noise”); 20100043796 (Meynink et al. 2010, “Systems for Reducing Exhalation Pressure in a Mask System”); 20100078016 (Andrieux et al. 2010, “Battery Management for a Breathing Assistance System”); 20100147302 (Selvaraj an et al. 2010, “Ventless Mask CPAP System”); 20100242967 (Burbank et al. 2010, “Sleep Apnea Therapy with Naso-Phyrangeal Bypass”); 20110270031 (Frazier et al. 2011, “System and Method for Airway Manipulation”); 20110270043 (McKenna 2011, “Air Movement Energy Harvesting with Wireless Sensors”); and 20110277765 (Christopher et al. 2011, “System for Providing Flow-Targeted Ventilation Synchronized to a Patient's Breathing Cycle”).

### SUMMARY OF THIS INVENTION

This invention can be embodied as a wearable, self-contained device that provides energy-efficient Positive Airway Pressure (PAP) to treat Obstructive Sleep Apnea (OSA) without requiring either a connection to a bedside air source or a continuous connection to an external power source. The device embodiment of this invention has two key elements. The first key element is a bolus airflow member that accumulates energy when there is a low probability of a person having an airway obstruction event and uses this accumulated energy to send a pressurized bolus of airflow into the person's airway when there is a high probability of the person having an airway obstruction event. The second key element is a primary airflow member that sends pressurized airflow into the person's airway in a more continuous and even manner than the bolus airflow member.

In this invention, the bolus airflow member and the primary airflow member can operate in parallel. Such parallel operation means that: airflow from the primary airflow member can be directed into the person's airway without going through the bolus airflow member; and airflow from the bolus airflow member can be directed into the person's airway without going through the primary airflow member. Operating together in a coordinated manner, the primary airflow member and the bolus airflow member provide just the right amount of pressure that is needed at any given time (not too little and not too much) in order to keep the person's airway open in the most energy-efficient manner.

The minimum amount of positive pressure that is required to keep a sleeping person's airway open can vary with different respiratory phases within a single respiratory cycle. This minimum required pressure can also vary with different respiratory cycles over the span of multiple respiratory cycles. Airway obstruction may occur in only a small percentage of respiratory cycles. This can allow for energy accumulation by a bolus airflow member over the span of multiple respiratory cycles before the accumulated energy is needed to create a bolus of airflow.

For these reasons, it can be energy efficient to provide different airflow pressure levels at different times. However, it can be energy inefficient to provide varying air pressure by iteratively increasing and decreasing the speed of an electric motor. Motors generally run more efficiently when they run at a relatively constant speed. The invention disclosed herein solves this problem. This invention can provide optimally-efficient variation in air pressure level with an electric motor that runs at a relatively-constant speed. The invention disclosed herein can also improve energy efficiency by harvesting the kinetic energy of exhalation. This invention can provide Positive Airway Pressure (PAP) in a more energy-efficient manner than is possible with devices in the prior art that have only one airflow member or devices that have two airflow members that do not operate in parallel.

Energy efficiency is particularly critical for wearable, self-contained Positive Airway Pressure (PAP) systems that a person can wear on their head without a tube connection to a bedside unit or a continuous wire connection to an external power source. Battery life is a major issue with such systems. A wearable, self-contained PAP system can be more convenient, less intrusive, and more portable than a traditional PAP system. This novel invention, which can increase the energy efficiency of wearable self-contained PAP systems, can make such systems more convenient and effective. This, in turn, can increase patient compliance with PAP therapy and improve OSA treatment outcomes. This offers a significant improvement over the prior art and its novel features are not anticipated by the prior art.

### INTRODUCTION TO THE FIGURES

FIGS. 1 through 12 show four examples (three sequential figures for each example) of how this invention can be embodied, but they do not limit the full generalizability of the claims.

FIGS. 1 though 3 provide a general example of how this invention can be embodied in a wearable device and method for energy-efficient Positive Airway Pressure (PAP) to treat Obstructive Sleep Apnea (OSA).

FIG. 1 shows a general example of how this device can operate during exhalation with a low probability of airway obstruction.

FIG. 2 shows a general example of how this device can operate during inhalation with a low probability of airway obstruction.

FIG. 3 shows a general example of how this device can operate during inhalation with a high probability of airway obstruction.

FIGS. 4 though 6 provide a specific example of how this invention can be embodied in a wearable device and method wherein energy is accumulated in a bolus airflow member using a separate electric motor in the bolus airflow member.

FIG. 4 shows this specific example during exhalation with a low probability of airway obstruction.

FIG. 5 shows this specific example during inhalation with a low probability of airway obstruction.

FIG. 6 shows this specific example during inhalation with a high probability of airway obstruction.

FIGS. 7 though 9 provide a specific example of how this invention can be embodied in a wearable device and method wherein energy is accumulated in a bolus airflow member using airflow from a primary airflow member.

FIG. 7 shows this specific example during exhalation with a low probability of airway obstruction.

FIG. 8 shows this specific example during inhalation with a low probability of airway obstruction.

FIG. 9 shows this specific example during inhalation with a high probability of airway obstruction.

FIGS. 10 though 12 provide a specific example of how this invention can be embodied in a wearable device and method wherein energy is accumulated in a bolus airflow member using airflow from a person's exhalation.

FIG. 10 shows this specific example during exhalation with a low probability of airway obstruction.

FIG. 11 shows this specific example during inhalation with a low probability of airway obstruction.

FIG. 12 shows this specific example during inhalation with a high probability of airway obstruction.

### DETAILED DESCRIPTION OF THE FIGURES

FIGS. 1 through 12 show four examples (three figures for each example) of how this invention can be embodied in a wearable device for providing energy-efficient Positive Airway Pressure (PAP) to treat Obstructive Sleep Apnea (OSA). These examples each include a bolus airflow member and a relatively-continuous primary airflow member, wherein these two airflow members can operate in a parallel and coordinated manner to provide just the right amount of positive airway pressure at any given time. These figures also show how this invention can be embodied in a method for providing energy-efficient Positive Airway Pressure (PAP). However, these figures show just some of the many examples of how this invention can be embodied. They do not limit the full generalizability of the invention claims.

FIGS. 1 though 3 provide an introduction to how this invention can be embodied in a wearable device and method for energy-efficient Positive Airway Pressure (PAP) to treat Obstructive Sleep Apnea (OSA). FIGS. 1 through 3 show the same device as it operates during three different respiratory phases or states: (1) exhalation with a low probability of airway obstruction, (2) inhalation with a low probability of airway obstruction, and (3) inhalation with a high probability of airway obstruction.

In an example, all three of these respiratory phases or states can occur at different times within each respiratory cycle. In an example, one or more of these respiratory phases or states may only occur only once during the span of multiple respiratory cycles. In an example, the state wherein there is a high probability of an airway obstruction event may only occur during a small percentage of respiratory cycles. In an example, this device can include a respiratory sensor to detect when there is a high probability that the person is having, or could have, an airway obstructive event during a particular respiratory cycle.

FIG. 1 shows this device operating during a time of exhalation when there is a low probability that the person is having, or could have, an airway obstruction event. FIG. 2 shows this device in operation during a time of inhalation when there is a low probability that the person is having, or could have, an airway obstruction event. FIG. 3 shows this device in operation during a time of inhalation when there is a high probability that the person is having, or could have, an airway obstruction event. In this example, the highest level of Positive Airway Pressure (PAP) is required in order to prevent airway obstruction during the phase or state represented in FIG. 3.

We now discuss in more detail the structural components of the device shown in FIGS. 1 through 3. FIG. 1 shows a cross-sectional view (looking down from the top of the person's head) of a respiratory mask 103 into which three airflow members have been integrated. In this example, mask 103 creates a mask interior space 104 that encloses the person's nose and nares 102. In an example, mask 103 can cover the person's mouth as well as the person's nose. FIG. 1 shows a cross-sectional view of a portion of the front perimeter of the person's face 101, including a cross-sectional view of the person's nares 102. The person's nares 102 are enclosed by the mask interior space 104 of mask 103, which is also shown in a cross-sectional view.

FIG. 1 shows two key airflow-creating members that operate in parallel: a bolus airflow member 105 and a more-continuous primary airflow member 110. In FIG. 1, bolus airflow member 105 accumulates energy during times when there is a low probability that the person is having, or could have, an airway obstruction event. It will use this accumulated energy later to send a bolus of airflow into the person's airway when there is a high probability that the person is having, or could have, an airway obstruction event. FIG. 1 introduces the general concept of a bolus airflow member 105. More specific details for selected embodiments of the bolus airflow member 105 are shown in later figures.

FIG. 1 also shows an air channel 106 that connects bolus airflow member 105 to ambient air through the exterior wall of mask 103 and connects bolus airflow member 105 to the mask interior space 104 through the interior wall of mask 103. FIG. 1 also shows two air valves, 108 and 107 that regulate fluid communication: between bolus airflow member 105 and ambient air; and between bolus airflow member 105 and mask interior space 104. In FIG. 1, both of these valves are shown as being closed, as represented by the “X” symbol that is drawn through their centers.

In an example, valves 107 and 108 can be electromagnetic valves which are opened or closed by solenoids which, in turn, are controlled by control unit 119. Technologies for opening and closing air valves, including any connecting wires or tubes, are well known in the prior art. Further, the precise opening and closing mechanisms are not central to this invention. Accordingly, in the interest of not cluttering up these figures, specific valve opening mechanisms (and connecting wires or tubes) are not shown in these figures.

FIG. 1 shows the operation of this device during exhalation when there is a low probability of an airway obstruction event. During this time, bolus airflow member 105 is accumulating energy for later creation of a bolus of airflow when it is needed to correct or prevent an airway obstruction. In FIG. 1, the accumulation of energy during this time is symbolically represented by a “lightning bolt” symbol 109 that is shown on bolus airflow member 105.

As we will discuss further in subsequent figures, energy can be accumulated and stored in bolus airflow member 105 in one or more forms selected from the group consisting of: tensile energy, kinetic energy, potential kinetic energy, electrical energy, pneumatic energy, and hydraulic energy. As we will also discuss further, energy can be accumulated and stored in bolus airflow member 105 using one or more members selected from the group consisting of: a coiled spring or other tensile member; a rotating impeller, turbine, fan, pinwheel, or flywheel; an electricity-producing generator and/or motor; a piezoelectric member; and a diaphragm, balloon, piston or other member that is displaced by air pressure.

FIG. 1 also shows a primary airflow member 110 that is also integrated into mask 103. Primary airflow member 110 creates positive pressure airflow in a more continuous and constant manner than does bolus airflow member 105. In this invention, primary airflow member 110 can operate in parallel with bolus airflow member 105. In this disclosure, parallel operation means that: airflow from primary airflow member 110 can be directed into the person's airway via mask interior space 104 without going through bolus airflow member 105; and airflow from bolus airflow member 105 can be directed into the person's airway via mask interior space 104 without going through primary airflow member 110. Parallel and coordinated operation of these two airflow members can provide greater design flexibility and energy efficiency than is possible with devices which have only one airflow member and devices with two airflow members which can only operate in series.

In an example, primary airflow member 110 can comprise a rotating air-moving member such as a turbine, fan, blower, or impellor. In an example, this rotating air-moving member can be rotated by an electric motor. There are numerous examples of electrically-powered air turbines, fans, blowers, and impellors in the prior art and the precise type of primary airflow member is not central to this invention. Accordingly, the precise type of primary airflow member is not detailed in these figures.

FIG. 1 also shows an air channel 111 that connects primary airflow member 110 to ambient air through the exterior wall of mask 103 and connects primary airflow member 110 to mask interior space 104 through the interior wall of mask 103. FIG. 1 also shows two air valves, 113 and 112 that regulate fluid communication: between primary airflow member 110 and ambient air; and between primary airflow member 110 and mask interior space 104. In FIG. 1, both of these valves are closed, as shown by their having an “X” drawn in their centers. In an example, valves 113 and 112 can be electromagnetic valves which are opened or closed by control unit 119. Since many valve-control technologies are known in the prior art and since the precise technology used is not central to this invention, specific valve opening mechanisms and connections (such as wires or tubes to the control unit) are not shown in these figures.

FIG. 1 also shows a separate air channel for exhalation. This separate air channel includes exhalation airflow member 114, air channel 115, and valves 116 and 117. In FIG. 1, during normal exhalation, valves 116 and 117 are open. In FIG. 1, dotted-line arrow 118 represents exhalation airflow that exits the mask through exhalation airflow member 114. In an example, exhalation airflow member 114 can be just empty space that neither resists nor accelerates exhalation airflow 118. In an example, exhalation airflow member 114 can include an air turbine, fan, blower, or impellor that can decelerate or accelerate exhalation airflow 118.

FIG. 1 also shows a control unit 119 which controls the operation of the device, including bolus airflow member 105, primary airflow member 110, exhalation airflow member 114, and air valves 107-108, 112-113, and 116-117. In an example, there can be wires or electronic circuits that connect control unit 119 with these airflow members and valves. However, the precise specification of such connecting wires or circuits is not central to this invention and would clutter up the figures. Accordingly, this figure does not show details concerning connecting wires or electronic circuits. Also, in an example, such connections could be wireless.

In an example, control unit 119 can include a sensor that monitors the person's respiration to identify the phases of respiratory cycles and/or detect possible airway obstruction events. In an example, control unit 119 can be in wireless communication with a respiratory sensor that is worn elsewhere on, or within, the person's body. In an example, control unit 119 can have electronic components and be programmable. In an example, control unit 119 can be integrated into the main body of a mask. In an example, control unit 119 can be in wireless communication with a remote control unit.

FIG. 1 also shows a power source 120. In an example, power source 120 can be a rechargeable battery, microchip, and/or capacitor. In an example, power source 120 can be recharged by a removable connection to an outside power source such as an electrical outlet, wherein this connection can be removed during operation of the device while the person sleeps. In an example, power source 120 can be recharged, in whole or in part, by generation of electricity from airflow passing through exhalation airflow member 114 and/or bolus airflow member 105.

In an example, power source 120 can provider power for control unit 119. The precise form of connection between power source 120 and control unit 119 is not central to this invention and is not shown, but could be as simple as a wire passing through the mask. In an example, power source 120 can power bolus airflow member 105 and/or primary airflow member 110 directly. In an example, power source 120 can power bolus airflow member 105 and/or primary airflow member 110 indirectly, via control unit 119. In an example, power source 120 can power air valves directly. In an example, power source 120 can power air valves indirectly, via control unit 119.

FIG. 2 shows the operation of the same device that was shown in FIG. 1, but during a different respiratory phase or state. FIG. 2 shows this device operating during inhalation when there is a low probability that the person is having, or could have, an airway obstruction event. In the example shown in FIG. 2, valves 116 and 117 have been closed by control unit 119 and exhalation airflow 118 has stopped. In this example, valves 112 and 113 have been opened by control unit 119. Inhalation airflow 201 is now passing into the mask through, and being accelerated by, primary airflow member 110, creating a pressurized airflow into the person's airway via mask interior space 104. In this example, airflow 201 is accelerated into the mask interior by a fan, blower, turbine, or impellor in primary airflow member 110 that is powered by power source 120 and controlled by control unit 119.

As shown in FIG. 2 by “lightning bolt” symbol 109, bolus airflow member 105 continues to accumulate energy during this respiratory phase and does not yet release a bolus of airflow because it is not yet needed. Even though FIG. 2 shows that inhalation is occurring, it does not show inhalation during a time when there is a high probability that the person is having, or could have, an airway obstruction event. Accordingly, an added bolus of air is not needed. The modest pressurized airflow 201 from primary airflow member 110 is therapeutically sufficient.

In an example, the respiratory phase or state shown in FIG. 2, wherein there is inhalation without a high probability of an airway obstruction event, can occur in the latter phase of inhalation, after an initial transition from exhalation to inhalation.

FIG. 3 shows the operation of this same device that was shown in FIGS. 1 and 2, but during a different respiratory phase or state. FIG. 3 shows this device operating during inhalation when there is a high probability that the person is having, or could have, an airway obstruction event. In the example shown in FIG. 3, not only is the primary airflow member 110 in operation creating airflow 201, but bolus airflow member 105 is creating airflow 301 as well. In FIG. 3, bolus airflow member 105 uses the energy that it has accumulated to release a bolus of airflow 301 into the person's airway in order to correct, or avoid, an airway obstruction event.

In FIG. 3, valves 107 and 108 have been opened by control unit 119. Also in FIG. 3, bolus airflow member 105 has been activated by control unit 119 to transduce accumulated energy into a bolus of airflow 301. Bolus of airflow 301 and more-continuous airflow 201 combine to provide extra positive air pressure when it is most needed to keep the person's airway open. This can be more energy efficient than having a single variable-speed airflow member in which the motor must repeatedly speed up and slow down.

FIGS. 4 though 6 show a more-specific example of how this invention can be embodied in a wearable device and method to provide energy efficient Positive Airway Pressure (PAP) to treat Obstructive Sleep Apnea (OSA). In the example shown in FIGS. 4 through 6, this device comprises a bolus airflow member that: accumulates energy in a coiled spring (that is wound by a small electric motor) when there is a low probability that the person is having, or could have, an airway obstruction event; and uses this accumulated energy to release a bolus of airflow into the person's airway when there is a high probability that the person is having, or could have, an airway obstruction event.

Similar to the three-figure series shown in FIGS. 1 through 3, the three-figure series shown in FIGS. 4 through 6 shows this device in operation during three different respiratory phases or states: exhalation with low probability of airway obstruction, inhalation with low probability of airway obstruction, and inhalation with high probability of airway obstruction. In an example, all three respiratory phases or states can occur during a single respiratory cycle. In an example, the third respiratory phase wherein there is a high probability of an airway obstruction event may only occur during a selected respiratory cycle over the span of multiple respiratory cycles.

FIG. 4 shows this device in operation during a time of exhalation wherein there is a low probability that the person is having, or could have, an airway obstruction event. FIG. 5 shows this device in operation during a time of inhalation wherein there is a low probability that the person is having, or could have, an airway obstruction event. FIG. 6 shows this device in operation during a time of inhalation wherein there is a high probability that the person is having, or could have, an airway obstruction event.

In the example that is shown in FIG. 4, bolus airflow member 105 is specifically comprised of a turbine 401, axle 402, small gear 403, large gear 404, coil spring 405, and electric motor gear 406. In FIG. 4, turbine 401 can accelerate (or decelerate) airflow through bolus airflow member 105. In various examples, a different rotating air-moving member, such as a fan, blower, or impellor, may be used instead of turbine 401. In this example, turbine 401 rotates around axle 402 which is centrally connected to small gear 403, such that turbine 401 and small gear 403 rotate together.

In this example, the teeth of large gear 404 can engage the teeth of small gear 403 when large gear 404 is moved into engaging proximity to small gear 403. Alternatively, in this example, the teeth of large gear 404 can engage the teeth of electric motor gear 406 when large gear 404 is moved into engaging proximity to electric motor gear 406. In FIG. 4, during energy accumulation, the teeth of large gear 404 are engaged with the teeth of electric motor gear 406.

In this example, large gear 404 is connected to one end of coil spring 405, such that coil spring 405 is wound up when large gear 404 rotates in one direction (such as clockwise rotation) and unwinds when large gear 404 rotates in the other direction (such as counter-clockwise rotation). In this manner, coil spring 405 can accumulate potential kinetic energy from the rotation of large gear 404 in one direction (such as clockwise rotation) and can release kinetic energy through the rotation of large gear 404 in the other direction (such as counter-clockwise rotation). In an example, large gear 404 can rotate around an axle that is roughly parallel with axle 402.

FIG. 5 shows the operation of the same device that was shown in FIG. 4, but during a different respiratory phase or state. FIG. 5 shows this device operating during inhalation when there is a low probability that the person is having, or could have, an airway obstruction event. In the example shown in FIG. 5, valves 116 and 117 have been closed by control unit 119 and exhalation airflow 118 has stopped. In this example, valves 112 and 113 have been opened by control unit 119. Inhalation airflow 201 is now passing into the mask through, and being accelerated by, primary airflow member 110, creating a pressurized airflow into the person's airway via mask interior space 104. In this example, airflow 201 is accelerated into mask interior space 104 by a fan, blower, turbine, or impellor in primary airflow member 110 that is powered by power source 120 and controlled by control unit 119.

FIG. 5 shows that bolus airflow member 105 continues to accumulate energy and does not yet release a bolus of airflow because it is not yet needed. Specifically, in this example, clockwise rotation of electric motor gear 106 continues the winding of coil spring 405 that was shown in FIG. 4. In this manner, the bolus airflow member 105 (comprising turbine 401, axle 402, small gear 403, large gear 404, coil spring 405, and electric motor gear 106) accumulates energy that can be used to release a bolus of air when needed to correct, or avoid, an airway obstruction event.

In an example, if coil spring 405 reaches a maximum coiled tension before a bolus of air is required, then electric motor gear 106 can be turned off. In another example, if coil spring 405 reaches a maximum coiled tension before a bolus of air is required, then the teeth of the electric motor gear 106 can be disengaged from the teeth of large gear 404 and large gear can be held in place (until an air bolus is needed) by a supplemental engagement member or brake mechanism.

FIG. 6 shows the operation of this same device that was shown in FIGS. 4 and 5, but during a different respiratory phase or state. FIG. 6 shows this device operating during inhalation when there is a high probability that the person is having, or could have, an airway obstruction event. In the example that is shown in FIG. 6, not only is the primary airflow member 110 in operation creating airflow 201, but the bolus airflow member 105 (comprising turbine 401, axle 402, small gear 403, large gear 404, coil spring 405, and electric motor gear 106) creates airflow 301 as well. In FIG. 6, bolus airflow member 105 uses the energy that it has accumulated in order to release a bolus of airflow 301 into the person's airway in order to correct, or avoid, an airway obstruction event.

In FIG. 6, bolus airflow member 105 has been activated by control unit 119 to transduce accumulated energy into a bolus airflow 301. Specifically, in FIG. 6, control unit 119 has moved large gear 404 away from engagement with electric motor gear 406 and into engagement with small gear 403. This latter engagement causes the unwinding of coil spring 405, which rotates large gear 404, which rotates (at a faster speed) small gear 403, which rotates turbine 401, which sends a bolus of airflow 301 into the person's airway through mask interior space 104. This bolus of airflow 301, combined with more-continuous airflow 201, provides extra positive air pressure when it is most needed to keep the person's airway open.

This design with a bolus airflow member and a primary airflow member working in parallel and with bolus airflow member accumulating energy until the bolus of airflow is needed can be more energy efficient than designs in the prior art. This design is especially useful for wearable, self-contained Positive Airway Pressure (PAP) systems which are not connected by a tube to a bedside unit or connected by a wire to an external power source.

FIGS. 7 though 9 show another specific example of how this invention can be embodied in a wearable device and method to provide Positive Airway Pressure (PAP). Like the example that was shown in FIGS. 4 through 6, the device shown in FIGS. 7 through 9 comprises a bolus airflow member that: accumulates energy in a coiled spring when there is a low probability that the person is having, or could have, an airway obstruction event; and uses this accumulated energy to release a bolus of airflow into the person's airway when there is a high probability that the person is having, or could have, an airway obstruction event.

However, unlike the example in FIGS. 4 through 6 wherein energy is accumulated in the bolus airflow member from a small electric motor, in the example in FIGS. 7 through 9 the energy that is accumulated in bolus airflow member 105 is transduced from airflow from primary airflow member 110. In an example, depending on the efficiency with which this energy is transduced, this can be more energy efficient than having a separate electric motor to power bolus airflow member 105.

In the example in FIGS. 7 through 9, primary airflow member 110 produces pressurized airflow continuously, but this airflow is diverted to bolus airflow member 105 during respiratory phases or states when it is not needed to keep the person's airway open. In an example, running primary airflow member 105 continuously at the same rate can be more energy efficient than speeding it up and slowing it down in order to achieve minimally-required positive airway pressure during different respiratory phases and states.

Further, a design that allows parallel operation of primary airflow member 110 and the bolus airflow member 105, with intermittent diversion of airflow from the primary airflow member to bolus airflow member 105, can be more flexible and energy efficient than a design in which the primary airflow member and the bolus airflow member always operate in series. For example, when a primary airflow member and a bolus airflow member operate in series, such that airflow from the primary airflow member must travel through a bolus airflow member to enter the person's airway, then one is not able to have time periods during which the primary airflow member sends airflow into the person's airway but the bolus airflow member is closed off from the person's airway.

Also, when a primary airflow member and a bolus airflow member always operate in series, such that airflow from the primary airflow member must travel through a bolus airflow member to enter the person's airway, then the primary airflow member has to overcome any back pressure or resistance from the bolus airflow member structures to reach the person's airway. These are some of the reasons why the parallel flow structure, with intermittent controllable flow diversion, that is shown in FIGS. 7 through 9 can be more energy efficient than having two flow members operate in series.

FIGS. 7 through 9 show this device in operation during three different respiratory phases or states: exhalation with low probability of airway obstruction, inhalation with low probability of airway obstruction, and inhalation with high probability of airway obstruction. In an example, all three respiratory phases or states can occur during a single respiratory cycle. In an example, the third respiratory phase wherein there is a high probability of an airway obstruction event may only occur during a selected respiratory cycle over the span of multiple respiratory cycles.

FIG. 7 shows this device in operation during a time of exhalation wherein there is a low probability that the person is having, or could have, an airway obstruction event. FIG. 8 shows this device in operation during a time of inhalation wherein there is a low probability that the person is having, or could have, an airway obstruction event. FIG. 9 shows this device in operation during a time of inhalation wherein there is a high probability that the person is having, or could have, an airway obstruction event.

The structure of the bolus airflow member that is shown in FIGS. 7 through 9 is like the structure shown in FIGS. 4 through 6, except that rather than having coil spring 405 be wound up by a small electric motor, coil spring 405 is wound up when turbine 401 is rotated by diverted airflow 703 from primary airflow member 110. FIG. 7 shows an airflow diversion channel 701 connecting air channel 113 (airflow output from primary airflow member 110) with air channel 106 (airflow input to bolus airflow member 105). FIG. 7 also shows a valve 702 that opens or closes diversion channel 701. In FIG. 7, valve 702 is open and there is diverted airflow 703 that exits primary airflow member 110 and enters bolus airflow member 105.

In an example, this device can divert airflow 703 from the primary airflow member 110 into the bolus airflow member 105 when there is low probability that the person is having, or could have, an airway obstruction event. In another example, the device can divert airflow 703 from the primary airflow member into the bolus airflow member whenever there is not a high probability that the person is having, or could have, an airway obstruction event.

As shown in FIG. 7, diverted airflow 703 rotates turbine 401, which rotates small gear 403, which rotates large gear 404, which winds up spring coil 405. In an example, large gear 404 can have a one-way ratchet mechanism that engages the teeth of large gear 404 to allow only one-way rotation during the accumulation of energy and disengages the teeth of large gear 404 to allow the release of energy from the coiled spring 405 to create a bolus of airflow when needed. In an example, movement of a one-way ratchet mechanism can be controlled by control unit 119. In an example, a ratchet mechanism may engage the teeth of large gear 404 to allow the spring to be wound up during times when there is a low probability that the person will have an airway obstruction event and may disengage the teeth of large gear 404 to allow the spring to unwind during times when there is a high probability that the person will have an airway obstruction event.

FIG. 8 shows the operation of this same device that was shown in FIG. 7, but during a different respiratory phase or state. FIG. 8 shows this device operating during inhalation when there is a low probability that the person is having, or could have, an airway obstruction event. In the example shown in FIG. 8, valve 702 has been closed by control unit 119 and diversion airflow 703 has stopped. Inhalation airflow 201 is now passing into the mask through, and being accelerated by, primary airflow member 110, creating a pressurized airflow into the person's airway via mask interior space 104.

FIG. 9 shows the operation of the same device that was shown in FIGS. 7 and 8, but during a different respiratory phase or state. FIG. 9 shows this device operating during inhalation when there is a high probability that the person is having, or could have, an airway obstruction event. In the example shown in FIG. 9, not only is the primary airflow member 110 creating airflow 201, but bolus airflow member 105 (comprising turbine 401, axle 402, small gear 403, large gear 404, and coil spring 405) creates airflow 301 as well. In FIG. 9, the bolus airflow member 105 uses the energy that it has accumulated in order to release a bolus of airflow 301 into the person's airway in order to correct, or avoid, an airway obstruction event.

The structure of the bolus airflow member in FIGS. 10 through 12 is like the structure shown in FIGS. 7 through 9, except that rather than having the coil spring wound by airflow from the primary airflow member, the coil spring is now wound from a portion of the exhalation airflow. In FIG. 10, valves 107 and 108 are open during exhalation, allowing portion 1001 of exhaled airflow to exit the mask through bolus airflow member 105. As shown in FIG. 10, exhaled airflow 1001 rotates turbine 401, which rotates small gear 403, which rotates large gear 404, which winds up spring coil 405. A one-way ratchet mechanism similar to that discussed for FIGS. 7 through 9 can be controlled by control unit 119 to regulate the accumulation and release of energy in bolus airflow member 105.

In this example, as shown in FIG. 11, energy is not accumulated in bolus airflow member 105 during any phase of inhalation, regardless of whether there is a low probability of an airway obstruction event or not. A potential disadvantage of harvesting energy from exhalation is that it may take longer to accumulate the amount of energy needed to provide a therapeutic bolus of airflow. A potential advantage of harvesting energy from exhalation is that it may be more energy efficient because it harvests some of the energy from exhalation airflow.

FIG. 12 shows how bolus airflow member 105 uses energy harvested from exhalation to create a bolus of airflow 301 when it is needed to correct, or prevent, an airway obstruction event. In an example, bolus of airflow 301 may not be needed during each respiratory cycle. In a example, bolus of airflow 301 may only be needed after multiple respiratory cycles which can give sufficient time for the transducing and accumulation of the required amount of energy from exhalation airflow.

As shown by the examples in FIGS. 1 through 12, this invention can be embodied in a device and method that includes a primary airflow member and a bolus airflow member. In an example, the primary airflow member can create a pressurized airflow which is sent into a person's airway. In an example, a primary airflow member can create positive pressure airflow using a fan, blower, turbine, or impellor that is powered by electricity. In an example, a primary airflow member can send positive pressure airflow into the person's airway more continuously than a bolus airflow member.

In an example, a primary airflow member can be located substantially within nine inches of the surface of a person's body. In an example, a primary airflow member can be located entirely within twelve inches of the surface of a person's body. In an example a primary airflow member can be integrated into a respiratory mask that a person wears over their nose and/or mouth. In an example, a primary airflow member can be worn on a person's head, separate from a mask, but in fluid communication with a mask. In an example, a primary airflow member can be in fluid communication with the interior of a respiratory mask and can send positive pressure airflow into a person's nose, mouth, or both, through the interior of this mask. In an example, a primary airflow member can be partly or entirely placed within a person's nasal cavity and/or oral cavity.

In an example, this device and method can also include a bolus airflow member that accumulates energy during a time when there is a low probability of the person having an airway obstruction event and uses this accumulated energy to create a bolus of pressurized air which is sent into a person's airway when there is a high probability of the person having an airway obstruction event. In an example, a bolus airflow member can accumulate and store energy for later use to create airflow when there is a high probability that the person is having, or could have, an airway obstruction event. In an example, this bolus airflow member can send a bolus of airflow into the person's airway only when needed to correct or prevent an airway obstruction event. In an example, this bolus airflow member can send a pressurized pulse of air into the person's nose, mouth, or both.

In an example, a primary airflow member and a bolus airflow member can operate in parallel. In an example, operating in parallel means that airflow from the primary airflow member can be directed into the person's airway without going through a portion of the bolus airflow member. This enables greater control over airflow and more efficient conversion of energy into variable pressure than if the primary airflow member and bolus airflow member were to only operate in series. In an example, operating in series means that airflow from the primary airflow member must go through a portion of the bolus airflow member in order to enter the person's airway.

In an example, a bolus airflow member can be located substantially within nine inches of the surface of the person's body. In an example, a bolus airflow member can be located within twelve inches of the surface of the person's body. In an example, a bolus airflow member can be in fluid communication with the interior space of a respiratory mask and can send positive pressure airflow into a person's nose, mouth, or both, through the interior space of this mask. In an example, a bolus airflow member can be integrated into a mask. In an example, a bolus airflow member can be worn on a portion of the person's head apart from, but connected to, a respiratory mask. In an example, a bolus airflow member can be partly or entirely placed within a person's nasal cavity and/or oral cavity.

In an example, a bolus airflow member can transduce, accumulate, and store energy when it is unlikely that the person is having, or could have, an airway obstruction event. In an example, a bolus airflow member can transduce this accumulated energy to create and send a bolus of air into the person's airway when it is likely that the person is having, or could have, an airway obstruction event.

In an example, the time when there is a high or low probability of the person having an airway obstruction event can be based on the phase of a person's respiratory cycle. In an example, the time when there is a high probability of the person having an airway obstruction can be at the onset of the inhalation phase of the respiratory cycle.

In another example, the time when there is a high or low probability of the person having an airway obstruction event can be based on the results from a sensor that monitors the person for possible airway obstruction events. In various examples, one or more sensors to monitor for airway obstruction events can be selected from the group consisting of: accelerometer, air pressure sensor, blood oxygen sensor, blood pressure sensor, body motion sensor, EMG sensor, other electrical activity sensor, gas analyzing sensor, heart rate sensor, piezoelectric sensor, pressure sensor, respiration sensor, other sound sensor, and strain gauge.

In an example, a bolus airflow member can transduce, accumulate, and store energy over the span of multiple respiratory cycles. In an example, a bolus airflow member can accumulate energy during multiple respiratory cycles so that it can be used when needed to correct or prevent an airway obstruction event. In an example, the bolus airflow member can accumulate energy during multiple respiratory cycles when positive pressure is not required to correct or prevent airway obstructions during these multiple respiratory cycles. In an example, a bolus airflow member can transduce energy that was accumulated over the span of multiple respiratory cycles into the creation of a bolus of airflow that is sent into the person's airway during a single selected respiratory cycle when there is a high probability of the person having an airway obstruction event.

In various examples, energy can be accumulated and stored in a bolus airflow member in one or more forms selected from the group consisting of: tensile energy, kinetic energy, potential kinetic energy, electrical energy, pneumatic energy, and hydraulic energy. In various examples, energy can be accumulated and stored in a bolus airflow member using one or more members selected from the group consisting of: a coiled spring or other tensile member; an impeller, turbine, fan, pinwheel, or flywheel; an electricity-producing generator and/or motor; a piezoelectric member; and a diaphragm, balloon, piston or other member that is displaced by air pressure.

In an example, a bolus airflow member can accumulate energy in the winding of a coiled spring (or other tensile member) and can create a bolus of airflow from the release of this energy as this spring (or other tensile member) unwinds. In an example a bolus airflow member can store potential kinetic energy by winding up a coiled spring. In an example, this coiled spring can create a bolus of airflow as it unwinds by spinning a turbine, fan, or blower.

In an example, energy can be stored in a spring by rotation of a turbine in one direction and can be released from the spring by rotation of the turbine in another direction. In an example, there may be a set of gears between the turbine and the spring so that there is a gear ratio between them. In an example, it can take multiple rotations of the turbine to rotate the center of the coil by one rotation. In an example, there can be a ratchet or catchment mechanism that is controlled by the device to allow the spring to wind up during certain times, to be held in place at other times, and to be released to unwind at other times.

In an example, a bolus airflow member can accumulate energy in the stretching of an elastic band (or other elastic member) and can create a bolus of airflow from the release of this energy as this elastic band contracts. In an example a bolus airflow member can store potential kinetic energy by stretching an elastic band. In an example, this elastic band can create a bolus of airflow as it contracts by spinning a turbine, fan, or blower. In an example, energy can be stored in the elastic band by rotation of a turbine in one direction and can be released from the band by rotation of the turbine in another direction. In an example, there may be a set of gears between the turbine and the elastic band so that there is a gear ratio between them. In an example, there can be a ratchet or catchment mechanism that is controlled by the device to allow the band to stretch during certain times, to be held in place at other times, and to contract at other times.

In an example, a bolus airflow member can accumulate energy in the acceleration of a flywheel and can create a bolus of airflow from the release of this energy as the flywheel decelerates. In an example, a bolus airflow member can store kinetic energy in a spinning flywheel. In an example, the flywheel can be accelerated by airflow leaving a mask and, when needed to correct or prevent an airway obstruction event, the flywheel can create airflow entering the mask as the flywheel decelerates. In an example, the direction of airflow through a turbine connected to a flywheel can be the same in both phases, but valves can be opened and closed to redirect the flow out of the mask in one phase and into the mask in another phase. In an example, a bolus airflow member can accumulate energy in the charging of a rechargeable battery, chip, or capacitor and can create a bolus of airflow from the discharge of electricity from this battery, chip, or capacitor. In an example, a battery can be charged by the rotation of a central axis in a generator, which is rotated by the rotation of a turbine, which is rotated by airflow through a channel. In various examples, this airflow can come from a primary airflow member and/or from the person's exhalation. In an example, the discharge of electricity from a battery can create airflow by rotating a turbine with an electric motor. In an example, the electric motor can be the same piece of hardware as the electric generator, but used in reverse. In an example, a bolus airflow member can accumulate energy in the inflation of a balloon (or other variable-volume air reservoir) and can create a bolus of airflow from the release of this energy as this balloon (or other variable-volume air reservoir) deflates. In an example, a bolus airflow member can store pneumatic energy in the form of air pressure within a balloon or other variable-volume air reservoir. In an example, inflation of the balloon can come from a portion of the airflow from a primary airflow member. In an example, some or all of the airflow from the primary airflow member can be directed into the balloon during periods of the respiratory cycle when airflow from the primary airflow member is not required to correct or prevent airway obstruction events.

In an example, a bolus airflow member can accumulate energy in the pressurization of a fixed-volume air reservoir and can create a bolus of airflow from the release of pressurized air when needed. In an example, pressurization of this reservoir can come from a portion of the airflow from a primary airflow member. In an example, some or all of the airflow from the primary airflow member can be directed into the reservoir during periods of the respiratory cycle when airflow from the primary airflow member is not required to correct or prevent airway obstruction events. In an example, a bolus airflow member can create a bolus of airflow as a fixed-volume air reservoir depressurizes.

In an example, a bolus airflow member can accumulate energy transduced from an electric motor. In an example, a bolus airflow member can comprise a spring or other mechanism that stores kinetic energy from an electric motor during the energy storing phase and transduces stored energy into airflow through a fan, blower, turbine, or impellor during the energy releasing phase.

In an example, accumulation of energy from a motor (such as in the form of a coiled spring, stretched elastic band, or inflated balloon) followed by its rapid conversion into a bolus of airflow can be more efficient and effective for creating a rapid bolus of airflow than direct generation of such a bolus by a variable-speed electric fan, blower, or impellor. In an example, a small, slow, low-power, constant-speed, and energy-efficient electric motor can gradually wind up a spring during exhalation or during multiple respiratory cycles when added positive pressure is not needed to correct or avoid an airway obstruction event. Then, when needed, the motor can be disengaged from the spring and the potential energy of the coiled spring can be rapidly transduced into a quick bolus of airflow via a fan, blower, or impellor that is in rotational communication with the spring (such as through a set of gears).

In an example, a bolus airflow member can accumulate energy transduced from (partial) airflow output from a primary airflow member. In an example, a bolus airflow member can transduce energy from the airflow output of the primary airflow member and use that stored energy to create airflow during a period of time when there is a high probability that the person is having, or could have, an airway obstruction event.

In an example, airflow from a primary airflow member can be redirected (by opening and closing valves) through a fan, blower, or impellor that is part of the bolus airflow member during certain respiratory phases or states. In an example, a portion of the airflow from the primary airflow member can be channeled through a fan, blower, or impellor that is part of the bolus airflow member on a substantively continuous basis. By harvesting some of the energy from airflow from the primary airflow member when less airflow is needed for therapeutic purposes, this device and method can be more energy efficient than having a second electric motor power a bolus airflow member.

By having a primary airflow member run at a relatively constant speed and by varying the portion of airflow that goes into the person's airway vs. the portion that goes through the bolus airflow member, this device and method can be more energy efficient than using a primary airflow member alone with a variable-speed airflow. Speeding up, or slowing down, an electric motor generally requires more energy than running one at a relatively constant speed. With the device and method disclosed herein, one can achieve the efficiencies of different pressure levels at different phases of the respiratory cycle without varying motor speed. One simply diverts different amounts of the primary airflow member's output to the bolus airflow member during different respiratory phases or states.

In an example, a bolus airflow member can accumulate energy transduced from airflow from the person's exhalation. In an example, some or all of the airflow from the person's exhalation can rotate a turbine as it exits a mask and this rotation can wind up a coiled spring. In an example, the potential kinetic energy stored in this spring can be released when needed to create a bolus of airflow to correct or prevent an airway obstruction event. In an example, rotation of a turbine by exhaled airflow can generate electricity and this electricity can be used later to create a bolus of airflow when needed. Having a primary airflow member and a bolus airflow member that can operate in parallel allows greater flexibility for harvesting energy from exhaled airflow than is possible if a primary airflow member and bolus airflow member always operate in series. This can enable greater energy efficiency. Energy efficiency is very important for wearable, self-contained Positive Airway Pressure (PAP) systems.

In an example, the operation of the primary airflow member and the bolus airflow member can be controlled by a control unit. In an example, a control unit can open and close valves to change the flow of air through either or both airflow members. In an example, a control unit can change the timing, duration, and/or volume of airflows that are created by the airflow members.

In an example, a control unit can be connected to both airflow control members by electricity-conducting wires or circuits. Connecting wires are well-known in the art and, in the interest of not cluttering up the figures, are not shown in the figures. One skilled in the art could easily figure out specific paths by which these components could be connected. In an example, these components could communicate via wireless communication. In an example, a control unit can be a remote unit that is in wireless communication with airflow members that are integrated into the wearable portion of a Positive Airway Pressure (PAP) system.

In an example, a control unit can control the accumulation of energy in the bolus airflow member over the span of multiple respiratory cycles and the release of the bolus of airflow from that accumulated energy only during a selected respiratory cycle when a sensor indicates that there is a high probability of the person having an airway obstruction event.

In an example, a control unit can enable someone to program one or more of the following factors: the timing, duration, efficiency, or amount of energy accumulated during a single respiratory cycle; the timing, duration, efficiency or amount of energy accumulated over the span of multiple respiratory cycles; the timing, duration, efficiency, amount, degree, mechanism, or form of energy use to create an airflow bolus; the timing, duration, efficiency, amount, degree, mechanism, or form of bolus to correct an airway obstruction event; the timing, duration, efficiency, amount, degree, mechanism, or form of bolus to prevent a predicted airway obstruction event; and the criteria for release of the airflow bolus.

FIGS. 1 through 12 also show how this invention can be embodied in a method to provide energy-efficient Positive Airway Pressure (PAP) to treat Obstructive Sleep Apnea (OSA). In an example, this invention can be embodied in a method comprising: (a) accumulating energy in a wearable bolus airflow member during a time when there is a low probability of the person having an airway obstruction event; (b) creating airflow into a person's airway continuously, or only during a time when there is a low probability of the person having airway obstruction event, using a wearable primary airflow member, wherein airflow from this primary airflow member can reach the person's airway without going through the bolus airflow member; and (c) using energy accumulated in the bolus airflow member to create a bolus of airflow into a person's airway when there is a high probability of the person having an airway obstruction event.

In an example, this invention can be embodied in a method of providing positive airway pressure comprising: (a) accumulating energy within a bolus airflow member during a time when there is a low probability of the person having an airway obstruction event, wherein the bolus airflow member is configured to be located substantially within nine inches of the surface of the person's body; (b) using energy accumulated within the bolus airflow member to create a bolus of pressurized air which is sent into a person's airway only when there is a high probability of the person having an airway obstruction event; and (c) and creating a pressurized airflow which is sent into a person's airway using a primary airflow member, wherein airflow from the primary airflow member can be directed to reach the person's airway without going through a portion of the bolus airflow member; and wherein the primary airflow member is configured to be located substantially within nine inches of the surface of the person's body.

In an example, this invention can be embodied in a wearable positive airway pressure device comprising: (a) a primary airflow member that creates pressurized airflow which is sent into a person's airway, wherein this airflow member is configured to be located substantially within nine inches of the surface of the person's body; and (b) a bolus airflow member that accumulates energy during a time when there is a low probability of the person having an airway obstruction event and uses this accumulated energy to send a bolus of pressurized air into the person's airway during a time when there is a high probability of the person having an airway obstruction event; wherein airflow from the primary airflow member can be directed into the person's airway without going through a portion of the bolus airflow member; wherein airflow from the bolus airflow member can be directed into the person's airway without going through a portion of the primary airflow member; and wherein the bolus airflow member is configured to be located substantially within nine inches of the surface of the person's body.

In an example, this invention can be embodied in a wearable positive airway pressure device comprising: (a) a primary airflow member that creates pressurized airflow which is sent into a person's airway, wherein this airflow member is configured to be located substantially within nine inches of the surface of the person's body; (b) a bolus airflow member that accumulates energy during a time when there is a low probability of the person having an airway obstruction event and uses this accumulated energy to send a bolus of pressurized air into the person's airway during a time when there is a high probability of the person having an airway obstruction event; wherein this bolus airflow member transduces energy accumulated over the span of multiple respiratory cycles into a bolus of airflow that is sent into the person's airway only during a selected respiratory cycle when there is a high probability of the person having an airway obstruction event; wherein airflow from the primary airflow member can be directed into the person's airway without going through a portion of the bolus airflow member; wherein airflow from the bolus airflow member can be directed into the person's airway without going through a portion of the primary airflow member; and wherein the bolus airflow member is configured to be located substantially within nine inches of the surface of the person's body; and (c) a control unit that controls the accumulation of energy in the bolus airflow member over the span of multiple respiratory cycles and the release of the bolus of airflow from that accumulated energy only during a selected respiratory cycle when a sensor indicates that there is a high probability of the person having an airway obstruction event.

In an example, a bolus airflow member can accumulate energy in the winding of a spring or other tensile member and create a bolus of airflow from the release of this energy as the spring or other tensile member unwinds. In an example, a bolus airflow member can accumulate energy in the stretching of an elastic member and create a bolus of airflow from the release of this energy as the elastic member contracts. In an example, a bolus airflow member can accumulate energy in the acceleration of a flywheel and create a bolus of airflow from the release of this energy as the flywheel decelerates.

In an example, a bolus airflow member can accumulate energy in the inflation of a variable-volume air reservoir and create a bolus of airflow from the release of this energy as the variable-volume air reservoir deflates. In an example, a bolus airflow member accumulates energy in the pressurization of a fixed-volume air reservoir and create a bolus of airflow from the release of this energy as the variable-volume air reservoir depressurizes. In an example, a bolus airflow member can accumulate energy in the charging of a battery, chip, and/or capacitor with electricity and create a bolus of airflow from the discharge of electricity from this battery, chip, or capacitor.

In an example, a bolus airflow member can accumulate energy transduced from an electric motor. In an example, a bolus airflow member can accumulate energy transduced from airflow from a primary airflow member. In an example, a bolus airflow member can accumulate energy transduced from airflow from the person's exhalation.

In an example, the time when there is a high probability of the person having an airway obstruction event can be based on one or more selected portions of the person's respiratory cycle. In an example, the time when there is a high probability of the person having an airway obstruction event can be based on results from a sensor that monitors to detect airway obstruction events.

In an example, a bolus airflow member can accumulate energy over the span of multiple respiratory cycles. In an example, a bolus airflow member can transduce energy accumulated over the span of multiple respiratory cycles into a bolus of airflow that is sent into the person's airway only during a selected respiratory cycle when there is a high probability that the person is having, or could have, an airway obstruction event.

In an example, a primary airflow member and a bolus airflow member can be worn outside the person's body. In an example, a primary airflow member and a bolus airflow member can be partially or completely placed within the person's nasal cavity and/or oral cavity. In an example, a control unit can allow adjustment of one or more of the following operational parameters: the timing, duration, efficiency, or amount of energy accumulated during a respiratory cycle; the timing, duration, efficiency or amount energy accumulated over the span of multiple respiratory cycles; the criteria for release of the airflow bolus; and the timing, duration, efficiency or amount of the airflow bolus.

In an example, this invention can be embodied in a method of providing positive airway pressure device comprising: (a) accumulating energy within a bolus airflow member during a time when there is a low probability of the person having an airway obstruction event, wherein the bolus airflow member is configured to be located substantially within nine inches of the surface of the person's body; (b) using energy accumulated within the bolus airflow member to create a bolus of pressurized air which is sent into a person's airway only when there is a high probability of the person having an airway obstruction event; and (c) and also creating a more-continuous pressurized airflow which is sent into a person's airway using a primary airflow member, wherein airflow from the primary airflow member can be directed to reach the person's airway without going through a portion of the bolus airflow member; wherein airflow from the bolus airflow member can be directed into the person's airway without going through a portion of the primary airflow member; and wherein the primary airflow member is configured to be located substantially within nine inches of the surface of the person's body.

I claim:

1. A wearable positive airway pressure device comprising:
a primary airflow member that creates pressurized airflow which is sent into a person's airway, wherein this airflow member is configured to be located substantially within nine inches of the surface of the person's body; and
a bolus airflow member that accumulates energy during a time when there is a low probability of the person having an airway obstruction event and uses this accumulated energy to send a bolus of pressurized air into the person's airway during a time when there is a high probability of the person having an airway obstruction event; wherein airflow from the primary airflow member can be directed into the person's airway without going through a portion of the bolus airflow member; wherein airflow from the bolus airflow member can be directed into the person's airway without going through a portion of the primary airflow member; and wherein the bolus airflow member is configured to be located substantially within nine inches of the surface of the person's body.

2. The bolus airflow member in claim 1 wherein this bolus airflow member accumulates energy in the winding of a spring or other tensile member and creates a bolus of airflow from the release of this energy as the spring or other tensile member unwinds.

3. The bolus airflow member in claim 1 wherein this bolus airflow member accumulates energy transduced from an electric motor.

4. The bolus airflow member in claim 1 wherein this bolus airflow member accumulates energy transduced from airflow from the primary airflow member.

5. The bolus airflow member in claim 1 wherein this bolus airflow member accumulates energy transduced from airflow from the person's exhalation.

6. The time when there is a high probability of the person having an airway obstruction event in claim 1 wherein this time is based on one or more selected portions of the person's respiratory cycle.

7. The time when there is a high probability of the person having an airway obstruction event in claim 1 wherein this time is based on results from a sensor that monitors to detect airway obstruction events.

8. The bolus airflow member in claim 1 wherein this bolus airflow member can accumulate energy over the span of multiple respiratory cycles.

9. The bolus airflow member in claim 1 wherein this bolus airflow member transduces energy accumulated over the span of multiple respiratory cycles into a bolus of airflow that is sent into the person's airway only during a selected respiratory cycle when there is a high probability that the person is having, or could have, an airway obstruction event.

10. The bolus airflow member in claim 1 wherein this bolus airflow member accumulates energy in the stretching of an elastic member and creates a bolus of airflow from the release of this energy as the elastic member contracts.

11. The bolus airflow member in claim 1 wherein this bolus airflow member accumulates energy in the acceleration of a flywheel and creates a bolus of airflow from the release of this energy as the flywheel decelerates.

12. The bolus airflow member in claim 1 wherein this bolus airflow member accumulates energy in the inflation of a variable-volume air reservoir and creates a bolus of airflow from the release of this energy as the variable-volume air reservoir deflates.

13. The bolus airflow member in claim 1 wherein this bolus airflow member accumulates energy in the pressurization of a fixed-volume air reservoir and creates a bolus of airflow from the release of this energy as the variable-volume air reservoir depressurizes.

14. The bolus airflow member in claim 1 wherein this bolus airflow member accumulates energy in the charging of a battery, chip, and/or capacitor with electricity and creates a bolus of airflow from the discharge of electricity from this battery, chip, or capacitor.

15. The primary airflow member and bolus airflow member in claim 1 wherein these members are worn outside the person's body.

16. The primary airflow member and bolus airflow member in claim 1 wherein these members are partially or completely placed within the person's nasal cavity and/or oral cavity.

17. A wearable positive airway pressure device comprising:
a primary airflow member that creates pressurized airflow which is sent into a person's airway, wherein this airflow member is configured to be located substantially within nine inches of the surface of the person's body;
a bolus airflow member that accumulates energy during a time when there is a low probability of the person having an airway obstruction event and uses this accumulated energy to send a bolus of pressurized air into the person's airway during a time when there is a high probability of the person having an airway obstruction event; wherein this bolus airflow member transduces energy accumulated over the span of multiple respiratory cycles into a bolus of airflow that is sent into the person's airway only during a selected respiratory cycle when there is a high probability of the person having an airway obstruction event; wherein airflow from the primary airflow member can be directed into the person's airway without going through a portion of the bolus airflow member; wherein airflow from the bolus airflow member can be directed into the person's airway without going through a portion of the primary airflow member; and wherein the bolus airflow member is configured to be located substantially within nine inches of the surface of the person's body; and
a control unit that controls the accumulation of energy in the bolus airflow member over the span of multiple respiratory cycles and the release of the bolus of airflow from that accumulated energy only during a selected respiratory cycle when a sensor indicates that there is a high probability of the person having an airway obstruction event.

18. The control unit in claim 18 wherein this control unit allows adjustment of one or more of the following operational parameters: the timing, duration, efficiency, or amount of energy accumulated during a respiratory cycle; the timing, duration, efficiency or amount energy accumulated over the span of multiple respiratory cycles; the criteria for release of the airflow bolus; and the timing, duration, efficiency or amount of the airflow bolus.

19. A method of providing positive airway pressure device comprising:
accumulating energy within a bolus airflow member during a time when there is a low probability of the person having an airway obstruction event, wherein the bolus airflow member is configured to be located substantially within nine inches of the surface of the person's body;
using energy accumulated within the bolus airflow member to create a bolus of pressurized air which is sent into a person's airway only when there is a high probability of the person having an airway obstruction event; and
and also creating a more-continuous pressurized airflow which is sent into a person's airway using a primary airflow member, wherein airflow from the primary airflow member can be directed to reach the person's airway without going through a portion of the bolus airflow member; wherein airflow from the bolus airflow member can be directed into the person's airway without going through a portion of the primary airflow member; and wherein the primary airflow member is configured to be located substantially within nine inches of the surface of the person's body.
